(19) United States
(12) Patent Application Publication
(10) Pub. No.: US 2020/0168304 A1
(43) Pub. Date: May 28, 2020

(54) CLINICAL TRIAL OVERSIGHT AND IDENTIFICATION OF ERRORS IN CLINICAL TRIAL PROCEDURE

(71) Applicant: MANA Consulting LLC d/b/a MANA RBM, Denver, CO (US)

(72) Inventor: Penelope K. Manasco, Denver, CO (US)

(21) Appl. No.: 16/777,792

(22) Filed: Jan. 30, 2020

Related U.S. Application Data
(63) Continuation-in-part of application No. PCT/US2019/013585, filed on Jan. 15, 2019.
(60) Provisional application No. 62/619,023, filed on Jan. 18, 2018.

(51) Int. Cl. G16H 10/20

(52) U.S. Cl. CPC G16H 10/20

(57) ABSTRACT

Deviations from a clinical trial protocol are identified based on automated analysis of clinical trial data. Clinical trial data can be collected for subjects or investigators at multiple study sites using any number of systems or collection methods. The types of data to be analyzed can be defined by identifying aspects of conducting a clinical trial which are imperative to maintaining integrity of the clinical trial data. Rules can be defined based on the identified critical aspects to establish criteria to be satisfied to ensure that the clinical trial has been conducted according to the protocol. A cross-database analysis of clinical trial data is performed to determine whether deviations have occurred. Deviations are logged for subsequent analysis to determine whether the deviation is part of a greater trend in errors. The associated clinical trial data can be further evaluated and the clinical trial workflow directed to address the identified errors.

### TECHNICAL FIELD

The application relates generally to clinical trial management conducting oversight of clinical trials and directing the workflow for conducting clinical trials.

### BACKGROUND

Clinical trials are conducted in accordance with a protocol which is developed for the clinical trial. A clinical trial protocol typically establishes, for instance, how the clinical trial will be conducted, ethical considerations and potential risks, and parameters for assessing the efficacy of the hypothesis underlying the clinical trial (e.g., primary and secondary efficacy endpoints). Deviations from the protocol when conducting a clinical trial may impact the integrity of the collected clinical trial data. Clinical trial data includes study subject specific data, or data collected for or related to clinical trial participants, and operational data, which can include data related to a study site and compliance with regulatory requirements. Both study subject specific data and operational data may be collected or logged using a number of distinct systems, such as an electronic data capture (EDC) system and an electronic diary (eDiary) system. Collected clinical trial data is thus distributed across repositories maintained for each of the systems utilized for data collection.

For any clinical trial, the veracity of the results is dependent upon the minimization of errors in procedure. To correct these errors, they must first be identified. In the current clinical trial paradigm, monitors conducting trial oversight review trial data within a data collector, which typically is an electronic data capture tool configured to collect data from an individual subject for each visit with simple logic checks. The oversight of clinical trial conduct routinely includes instances of monitoring within confined areas, or “silos.” In other words, traditional monitoring is performed by a range of people within different functional areas, where each of these functional areas can be limited to its own “silo:” clinical monitoring (often called monitors or clinical research associates or CRAs), data management, medical/safety monitoring, and project management, for example.

### BRIEF DESCRIPTION OF THE FIGURES

FIG. 1 illustrates a clinical trial oversight system corresponding to example embodiments of the present disclosure.

FIG. 2 illustrates an example method performed by a clinical trial monitoring entity according to one or more embodiments.

FIG. 3 illustrates details of an example computing device and clinical trial monitoring entity according to one or more embodiments.

FIG. 4 depicts an example conceptual diagram of identifying deviations from a clinical trial protocol based on collected clinical trial data.

FIG. 5 depicts an example conceptual diagram of analyzing identified clinical trial protocol deviations to identify errors in conducting the clinical trial.

FIG. 6 is a flowchart of example operations for determining critical aspects for conducting a clinical trial in accordance with a clinical trial protocol.

FIG. 7 is a flowchart of example operations for identifying deviations from a clinical trial protocol based on analysis of collected clinical trial data.

FIG. 8 is a flowchart of example operations for identifying high impact and systematic errors in conducting a clinical trial based on identified protocol deviations.

FIG. 9 is an example conceptual diagram of performing quality evaluation for an entity involved in a clinical trial based on collected clinical trial data.

FIG. 10 is a flowchart of example operations for performing a quality evaluation of an entity involved in a clinical trial based on one or more monitoring criteria established for the clinical trial.

FIG. 11 is a flowchart of example operations for creating rules for identifying events and/or deviations from a standard established for a clinical trial.

FIG. 12 is a flowchart of example operations for performing oversight of a clinical trial across sites of the clinical trial.

FIG. 13 is a flowchart of example operations for identifying systematic errors in compliance with one or more standards established for conducting a clinical trial.

FIG. 14 depicts an example computer system with a clinical trial oversight system.

### DETAILED DESCRIPTION

The present disclosure describes example techniques for identifying and correcting errors (e.g., data errors, systematic errors, etc.) in clinical trial processes and directing the workflow for conducting clinical trials in accordance with one or more specific studies and/or study types. In an aspect, these techniques can be independent of any particular data collector, clinical trial protocol, or data source, though the directives for identification and correction of errors can be study-specific. Although the techniques may involve performing customized analytics designed to identify errors in data documenting critical efficacy, safety, human subject protection, investigational product management, and Good Clinical Practice processes for a specific trial, the results and directives produced by the proposed system are not, in most cases, standard key performance indicators. Instead, the proposed system and techniques are designed to evaluate data from individual subjects and collection sites, across time and across datasets, to identify protocol-specific and systematic errors occurring at particular sites and to produce data visualizations and reports that can be utilized to set directives for correction of the identified errors.

### Introduction

Though monitoring these different functional areas separately can allow for error correction inside a particular silo, it does not allow for identification of broader errors in process or trends that span multiple functional areas, data systems, or data sets. Therefore, greater trends that can expose errors in critical trial data and process may not be identified. With this in mind, for purposes of the present disclosure, the term “monitoring” or “to monitor” in the context of the presently disclosed techniques is not limited in the way that traditional clinical trial monitoring is understood—namely, though monitoring according to aspects of the presently disclosed techniques can involve one or more monitors monitoring different functional areas (the silos) individually, it can also involve monitoring greater trends that cut across these individual silos or data systems or datasets. Similarly, the term “oversight” or “to oversee” can involve instances of one or more individuals monitoring the clinical trial according to the definition above, though such monitoring is not to be understood herein as constituting the only function performed during clinical trial oversight.

Likewise, current techniques for clinical trial oversight are not equipped to direct the workflow of the study to correct actions of study participants, data collection sites, and other participants to address identified errors, and to do so in accord with one or more specific studies or study types. In addition, urgent information may not be sent to remote study sites and oversight personnel in response to near real time review. This includes an inability to send notifications of missing data or critical data findings (e.g., findings generated based on application of one or more algorithms to the clinical trial data) and/or to generate a comprehensive list of activities across different data systems and different data types pertinent to directing workflow according to the one or more specific studies or study types.

In addition, with an ever-changing regulatory landscape that can introduce new regulatory compliance requirements at any time, quality control has become a critical aspect for clinical trial data oversight systems. Existing systems lack the ability, for example, to identify a random number of subjects, study team members, data collection sites, and/or review sites for quality control (QC) review/audit (or on which to perform other aspects of clinical trial oversight), nor do they provide reliable mechanisms for documenting that QC review. Moreover, current clinical trial oversight systems do not have the ability to identify critical data that is specific to one or more studies or study types and/or process errors. Moreover, in either case, existing systems lack the ability to perform these functions independent of a human monitoring team.

Overview

Clinical trial monitoring and oversight is conventionally performed through manual evaluation of collected clinical trial data. However, due to the volume of clinical trial data and the large number of disparate data systems which are collected for multiple subjects and study sites and are stored in different repositories based on the type of system used for data collection or entry, critical errors may not be detected, thus affecting the integrity of the collected data. Failure to promptly identify critical errors may also place clinical trial participants at risk.

To facilitate clinical trial oversight through detection of errors in how a clinical trial has been conducted which could impact clinical trial integrity, deviations from the aspects defined in a clinical trial protocol, applicable laws, or regulatory documents are identified based on automated analysis of clinical trial data. The clinical trial data can be collected for subjects or investigators at one or more study sites using any number of systems or collection methods. The types of data which are to be analyzed can be defined based on identifying aspects of conducting a clinical trial which are imperative to maintaining integrity of the clinical trial data in view of the protocol to facilitate selective review of clinical trial data which are pertinent to assessment of clinical trial protocol adherence. Rules can be defined based on the identified critical aspects to establish criteria for clinical trial data that are to be satisfied to ensure that the clinical trial has been conducted in accordance with the protocol. An automated cross-database analysis of clinical trial data can then be performed to determine whether deviations from the protocol have occurred and notifications sent to appropriate study staff upon identifying a deviation. Clinical trial data can be retrieved and analyzed periodically during a clinical trial to ensure that protocol deviations can be promptly identified and resolved.

Upon identifying a deviation from the protocol based on the analysis of clinical trial data, the deviation can be logged for subsequent analysis to determine whether the deviation is part of a greater trend in errors in conducting the clinical trial, such as errors identified consistently for an individual or across study sites. The clinical trial data and associated processes associated with the deviations for an individual, across multiple individuals, or across study sites can be further evaluated and corrected to prevent jeopardization of the clinical trial as a whole due to utilizing or reporting data which were not collected in compliance with the protocol. The clinical trial workflow can then be directed to address the reasons or “root cause” of the identified errors based on the results of the analysis and corrective actions can be undertaken. Deviations can be logged as issues for further analysis or for establishment of a corrective and preventative action plan (CAPA) to address the underlying error and prevent the error from recurring.

### Example Illustrations

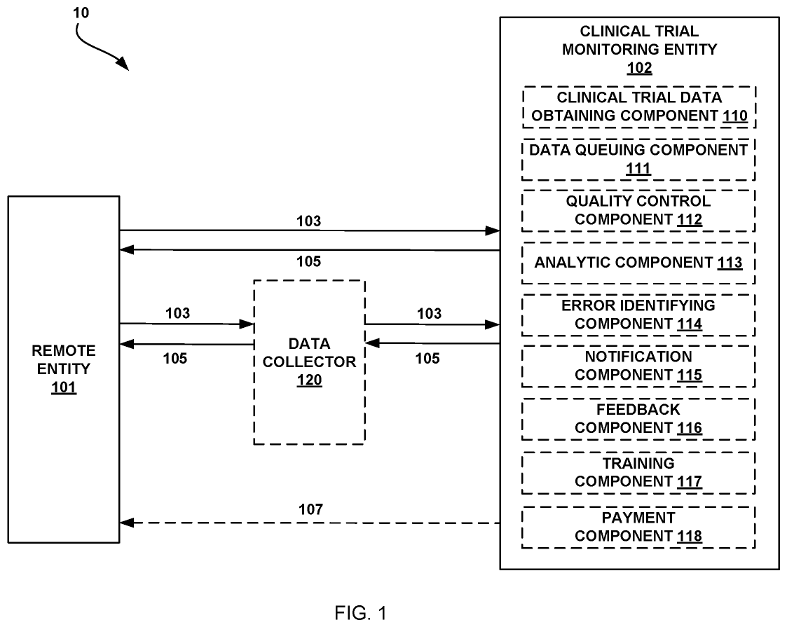

FIG. 1 illustrates an example system 10 for implementing the techniques introduced above. As shown, the example system 10 includes one or more remote entities 101 that can collect clinical trial data 103, for instance, from a group of test subjects and communicate the collected clinical trial data 103 to a data collector 120 and/or a clinical trial monitoring entity 102. The one or more remote entities 101 can include but is not limited to clinical trial subjects, collection sites where clinical trial data 103 is obtained from the trial subjects, collection site staff that can collect the clinical trial data 103 from the trial subjects, or any other person, device, or entity that can input or otherwise communicate the clinical trial data 103 to the data collector(s) 120 and/or clinical trial monitoring entity 102. For instance, remote entities 101 can also include clinical trial and/or site staff such as monitors, data reviewers, data managers, project managers, medical monitors, investigators, study coordinators, or research subjects. Clinical trial data 103 can include, but is not limited to, data related to or concerning a clinical trial, including data collected by one or more remote entities 101 before, during, or after a clinical trial. For instance, in one or more embodiments, clinical trial data 103 includes collected informed consent documentation, vital signs, physical exam or other clinical assessments, subject or patient reported outcomes or diaries, laboratory assessments, third party assessment of outcomes or radiologic findings, investigational product administration, output from biomonitoring systems, etc. Additionally or alternatively, clinical trial data 103 includes metadata associated with collection of data and queries (e.g., audit trail, monitoring oversight status). Additionally or alternatively, clinical trial data 103 includes delegation of authority or responsibility to personnel managing the clinical trial process (e.g., assignment of activities to staff by a principal investigator). Additionally or alternatively, clinical trial data 103 includes operational data such as investigational product receipt, processing, and destruction, training records, delegation of authority, and monitoring reports. Additionally or alternatively, clinical trial data 103 includes receipt, storage, preparation or return of investigational materials related to the clinical trial process (e.g., materials for a placebo, drug, device, vaccine, or other intervention administered in the clinical trial). Additionally or alternatively, clinical trial data 103 includes information regarding study or site personnel or participants of the clinical trial (e.g., training or subject information).

The clinical trial monitoring entity 102 is configured to obtain the collected clinical trial data 103 from one or more of the remote entities 101 and/or one or more data collectors 120 (e.g., using the Clinical Trial Data Obtaining Component 110). For instance, the clinical trial data (103) can be obtained from multiple remote entities (101) indirectly via the one or more data collectors 120 (e.g., different data collectors associated with different remote entities). In an aspect, the clinical trial data 103 can be obtained by importing it directly or manually from one or more data collectors 120, but may be limited in some examples according to data rules that a user may utilize to filter obtained clinical trial data 103. A user as used herein includes, but is not limited to, a user of an entity of the system 10 or an entity of the system 10 (e.g., remote entities 101, a clinical trial monitoring entity 102 or data collector 120). The clinical trial data 103 obtained by the clinical trial monitoring entity 102 can be obtained from any type of data collector 120 and in any form, including data arranged and/or stored at a data collector 120 according to any clinical trial data standard. The data is not limited to the actual values in the data collector. It also includes audit trail data describing who entered the data, when, and changes to the data. Additionally, it can also include query data, monitoring status, or other metadata from the data collectors. This can include, but is not limited to, clinical trial data 103 obtained according to the following techniques, technologies, and/or standards:

Electronic Data Capture (EDC): The EDC obtains, for instance, clinical trial data electronically (e.g., to support protocol compliance, study endpoints, and safety). Collected EDC clinical trial data includes metadata such as an audit trail (e.g., which staff or site member completed assessments, entered time, and/or entered reasons for change) and status of review as examples. Collected EDC clinical trial data also includes data on the queries raised within the EDC.

Electronic Source System: The electronic source system obtains, for instance, a subset of clinical trial data collected by the EDC. Clinical trial data from the electronic source system could include, for instance, the same components listed with respect to the EDC. Clinical trial data can be collected de novo in the electronic source system.

Electronic Patient Reported Outcome System (ePRO): The ePRO system obtains, for instance, clinical trial data collected from subjects to determine the effectiveness of an intervention or other outcome measure. Data collected from subjects includes the actual values for the assessments, and includes outcomes collected from study investigators. Clinical trial data also includes metadata from the ePRO system such as an audit trail and status of review as examples. Clinical trial data also includes data on the queries raised within the ePRO system if applicable.

Electronic Diary (eDiary): An eDiary includes electronic clinical trial data collected from research subjects or patients covering many aspects of their health, investigational product compliance, side effects, or hospital visits (e.g., prior to or during a clinical trial). This clinical trial data also includes metadata such as an audit trail and status of review as examples.

Electrocardiogram (ECG or EKG): An ECG or EKG obtains, for instance, electronic clinical trial data on cardiac function and physician interpretation which can include single measures or continuous cardiac monitoring, including physician assessment and metadata associated with the collection of the data.

Electronic Informed Consent (eConsent) system: An eConsent system obtains, for instance, data for clinical trial participants confirming both the date of informed consent, who signed it, versions, sub-study participation, withdrawal of consent, and the processes associated with the informed consent process.

Integrated Voice Response System (IVRS): AN IVRS obtains, for instance, electronic clinical trial data for clinical outcomes provided by patients via phone lines, data on treatment assignment including unblinding, and investigational product management, storage, and distribution and the metadata and queries associated with the data.

Integrated Web Response System (IWRS): AN IWRS obtains, for instance, electronic clinical trial data for clinical outcomes provided by patients, data on treatment assignment including unblinding, and investigational product management, storage, and distribution and the metadata and queries associated with the data.

Clinical Trial Management System (CTMS): CTMS obtains, for instance, electronic clinical trial data on site performance, operational data documenting trial conduct and oversight (e.g., monitoring visit reports), training, investigational product management, and any other operational aspect of a clinical trial. In addition to electronic data, this can include metadata associated with the data including but not limited to the audit trail.

Site Tracker Analyzing Risk (STAR) by MANA RBM of Denver, Colo. STAR obtains clinical trial data such as operational oversight, quality oversight, issue management, action items, training, technology access, delegation of authority, investigational product management, monitoring reports, and site feasibility data.

Electronic Trial Master File and Electronic Investigator Site File System (eTMF/eISF): An eTMF/eISF obtains data and/or metadata on the status of documents, their review in the eTMF/eISF, and/or presence or absence of critical essential documents.

Any type of bio-tracker (including, but not limited to, trackers to analyze blood glucose/blood sugar, heart rate, respiratory rate, activity). Bio-trackers include wearable devices worn to collect and/or analyze biological information (e.g., a heart monitor worn to collect and/or analyze heart rate). Bio-trackers can also be non-wearable devices to collect and/or analyze biological information (e.g., blood test strips).

Any type of laboratory results such as clinical data from electronic data with laboratory results, pharmacokinetic analyses, status of samples, errors in sample handling, discrepancies between expected and actual samples, and the metadata associated with any laboratory results.

Any type of electronic data from third party vendor assessments such as central radiologic reviewers including the metadata associated with the assessments.

Text responses/Text entry/Direct entry or other data and metadata associated with data provided via text messages.

Risk Assessment including risk identification, categorization, prioritization, mapping of oversight plans to risks, whether the risk occurred, and the metadata associated with the Risk Assessment.

Correction and Prevention Plan (e.g., remediation plan) (CAPA) including electronic data linking the CAPA to an issue, interventions, and outcomes of the interventions and the metadata associated with the CAPA.

Furthermore, the clinical trial data 103 can be used for any type of research or clinical trial, including, but not limited to: human (including, but not limited to biologics, small molecule, proteins, vaccines, gene therapy, medical device studies), plant studies, and animal studies, or any other type of research or clinical trial known in the art. The clinical trial data 103 can be used with multiple types of trials, including but not limited to: placebo-controlled studies, active-control studies, open-label studies, observational studies, epidemiology studies, pharmacokinetic studies, safety studies, virtual studies, or any other type or research or clinical trial known in the art. The clinical trial data 103 can be used for regulated and non-regulated studies (i.e., is not submitted to a regulatory agency for marketing approval or support of claims or safety e.g., studies by the National Institutes of Health for standard of care). In the non-limiting examples where the clinical trial data 103 is used for regulated studies, it can be used for any phase of such studies, including phases I, II, III, and/or IV or similar classifications.

In addition, the clinical trial monitoring entity 102 can include a data queuing component 111 that can provide access to collected data for reviewers of the data worldwide who have been granted access to the system 10. This allows for individual reviewers having appropriate system permissions to review the clinical trial data 103 collected from one or more remote entities 101. Depending on a given system implementation, this review can involve reviewing data at a broad, system level, while in other examples, the review can involve reviewers studying clinical trial data input by or for individual clinical trial subjects at a shared data collection site or even at their home or place of business.

Furthermore, the clinical trial monitoring entity 102 can include a quality control component 112 that allows oversight of individual reviewer activities (for instance, based on the queued data that has been reviewed). This functionality includes identifying reviews done by a specific monitor or reviewer or by a specific research entity (study site or study team functional group such as medical monitor, data management), and can be based on selection of a random list of subjects based on a percentage of subjects and/or data collection sites that are to be reviewed (e.g., 5%, 10%). Other aspects of performance can also be incorporated beyond subject review. For instance, the QC component 112 can also evaluate other aspects of site or study performance other than subject data such as documentation associated with regulatory requirements (e.g., financial disclosure, 1572, delegation of authority, investigational product management, training, or access to technology systems). In addition, the QC component 112 can be configured to document and oversee findings and their subsequent resolution (including issue management and Corrective and Preventative Action Plan management) or to push these findings to other reviewers, data collectors 120, and/or clinical trial monitoring entities 102 that may exist worldwide.

Based on the obtained clinical trial data 103, the clinical trial monitoring entity 102 can be configured to generate analytic data by applying the obtained clinical trial data 103 to one or more algorithms at an analytic component 113. In an aspect, the analytic component 113 can implement an analytic scaffold by importing one or more customized analytic algorithms or generating the algorithms within the analytic component 113 that can be utilized to identify potential errors produced in particular trials and by particular data collection sites or reviewing entities. Thus, the generated analytic scaffold applied by the analytic component 113 can be study-specific, which for purposes of the present disclosure, can mean customized to specific studies (e.g., protocols associated with the studies) and/or tailored and scheduled to produce reports (or data visualizations) and/or directives to correct the identified issues in the clinical trial procedure and direct workflow of study conduct in accord with a specific study or study type. For instance, in some examples, this may mean that the reports, data visualizations, algorithms, directives, or the like can be tailored to a particular subject, data collection site, to a study based on data from a particular set of subjects, or from any other possible error injection point along the life cycle of the clinical trial or trials. In addition, although embodiments described herein may be described as “study-specific” in nature, this is not meant to be a limiting aspect. Instead, more general (i.e., non-study-specific) reports, findings, analytic data, etc. can also be utilized in some example embodiments of the system, methods, and devices.

Additionally, or alternatively identifying errors can be based on risk assessment. For instance, the analytic component 113 can implement an analytic scaffold by importing one or more customized analytic algorithms or generating the algorithms within the analytic component 113 based on risk assessment. For instance, risk assessment can involve, performing identification, categorization (e.g., categorizing as high or low risk) or prioritization (e.g., determining which clinical trial data, errors, or risk are most important); mapping of oversight plans to risks; determining whether a risk occurred; etc. In one or more aspects of embodiments, one or more customized analytic algorithms filter or weight obtained clinical trial data based on risk assessment (e.g., based on a study specific risk assessment). Furthermore, once the analytic data is produced by the analytic component 113, an error identifying component 114 can be configured to review the analytic data to identify one or more errors and trends in errors in the clinical trial process. This may include, for instance, locating one or more deviations in the analytic data (or clinical trial data 103) from a configured threshold value that serves as a baseline for particular analytic data values (e.g., values averaged across data collected over time for multiple collection sites for a particular analytic scaffold, trial, report paradigm, etc.). Alternatively or additionally, these errors can be defined in terms of a research plan or protocol associated with a particular trial or study. In addition, this review of the analytic data (or clinical trial data 103) can be continuous, such that the deviations from threshold values and associated errors can be identified in real time or near real time.

In an additional aspect, the clinical trial monitoring entity 102 can include a notification component 115 that is configured to provide feedback 105 that includes one or more notifications to an entity associated with the trial based on review of the analytic data (e.g., responsive to identification of an error by error identifying component 114). Depending on the implementation or scenario, the notified entity can be any research team member, clinical trial subject, reviewer or review site, investigative site, or any person or entity associated with the clinical trial, any of which may be or may not be associated with a remote entity 101.

In some examples, the notification component 115 can produce and deliver feedback 105 in the form of an immediate or nearly immediate text, audio, video, image, email or action item or any other form of notification that relays determined errors or issues to the notified entity or entities (e.g., a subject did not meet a threshold criteria). Alternatively or additionally, the notification can be a periodic (e.g., daily, weekly, monthly, yearly, etc.) notification of critical activities or action items (i.e., “to-do” activities). These critical activities or action items can be generated automatically as feedback 105 resulting from an identified issue or error, thereby incorporating the clinical trial data 103 and the generated analytic data. In an aspect, feedback component 116 can be configured to formulate these action items as one or more directives that can be tailored to a corrective or action plan configured specifically for a particular study, study type, trial, collection site, trial subject, or any other notified entity. This feedback 105 can be utilized to direct workflow for the clinical trial process in accordance with the protocol such that any identified errors or issues can be rectified, and can be corrected according to a specific schedule if applicable (e.g., correction upon receipt of an identified error or issue). For instance, feedback 105 can be used to direct workflow by identifying if a subject can be enrolled in a clinical trial (e.g., is the subject eligible for treatment) before performing the clinical trial process (e.g., instituting treatment), identifying that an error has occurred, or identifying that errors in process which were previously identified are resolved.

The action items in notifications can be auto-populated from the clinical trial monitoring entity 102 and or data collectors 120 in communication with the clinical trial monitoring entity 102. In some examples, gamification components can be incorporated into the feedback 105 to encourage and enable users to map their progress on critical activities for the specific study. For instance, the notifications can include completed actions (e.g., notifications of completion of training or training milestone) or a reward for completion of a completed action.

In addition, the feedback component 116 can be configured to generate one or more reports (or data visualizations) based on the analytic data, and can send the reports to one or more remote entities 101 and/or data collectors 120 as feedback 105 (such as, but not limited to, making them available through a web portal or application). The generated reports can serve as a documentation function so as to confirm monitor or reviewer activity or specific task and that it was completed on a specific date and/or time. These generated reports can direct study workflow (e.g., confirm that a research subject meets criteria for enrollment). In addition, the reports can log the performance of reviewers (e.g., relative to a particular review or study plan) based on the number of subjects and quality analysis, the performance of sites based on review/analytic data, deviations, trends, and other findings from the clinical trial monitoring entity 102 and the system 10, generally.

The feedback 105, including the notifications, to-dos, action items, analysis results, reports, data visualizations, etc., can be sent by any electronic form, such as text message, email, instant message, web or application custom notification, or the like.

In addition, clinical trial monitoring entity 102 can include a training component 117 configured to manage an electronic training program that trains users regarding how to conduct their role in a clinical trial (e.g., review of one or more specific clinical trials and/or clinical trial types). In some examples, the training may be specific to a role of a trained user, such as for a project manager, a remote reviewer, or the like. In an aspect, the training component 117 may be configured to store the training materials used for a particular user and/or role. In addition, for a particular user, the training component 117 can document the training completed for a specific role, for a particular type of review (e.g., data, medical, clinical, QC) and for a specific trial or trial type.

Unlike current clinical trial procedures where specific reviewers or monitors are assigned to oversee a specific trial, the clinical trial monitoring entity 102 and other devices and techniques described herein can allow a relatively large number of reviewers situated across the globe to access the clinical trial data and/or analytic data for review. In effect, by providing this data to reviewers via the Internet, wireless access networks, and other modern forms of information transmission and communication, a more geographic and temporal-independent clinical oversight and monitoring paradigm can be realized.

In an additional aspect, clinical trial monitoring entity 102 can include a payment component 118 configured to facilitate payment 107 to remote entities 101 (e.g., payment to reviewers based on completing review of trial data). In an aspect, payment component 118 can be configured to obtain, from one or more users/reviewers, payment information, tax information, and the like, and based on this obtained information, the payment component 118 can be configured to automatically pay one or more remote entities 101, for instance, when successful review has been completed. The payment component can include algorithms that can generate a payment amount per review or other scope of work that can be published for reviewers to determine which project they would like to perform review. Other aspects of facilitating payment 107 to remote entities 101 can include, but is not limited to, paying vendors or research sites for providing research data and/or materials, or paying research subjects for their participation in a clinical trial or study.

The traditional process for reviewing clinical trial data at each data collector on a page-by-page basis (in many cases without a focus on the critical data and the processes for collecting the data in a trial) can be improved by implementing the aspects described above. Specifically, the embodiments presented herein provide an improvement over the traditional process by automating, simplifying, centralizing, and coordinating a previously more fragmented and cumbersome clinical trial review, while still ensuring a comprehensive, integrated oversight of critical trial data. As a result, review of the resulting clinical trial data no longer entails piecemeal onsite visits to each of the individual research sites. Instead of this legacy paradigm, when utilizing the proposed system 10, trained monitors/reviewers can perform reviewing and corrective functions world-wide (e.g., at multiple locations) and at any time. Therefore, the techniques described herein can standardize, measure, perform, and report quality oversight with respect to the monitors/reviewers in each study, as well as the subjects of the study and the data collection sites that collect the raw clinical study data.

Furthermore, whenever possible, identification and notification of data errors can be done automatically (i.e., once the analytic configuration for a trial is complete). This assures critical deviations to the protocol are immediately identified, documented, reported, and used to review the performance of each research site and the study conduct as a whole. Moreover, the system 10 provides a synthesized proactive notification system for informing users of issues or actions, allowing users to quickly and efficiently respond to process errors without the cumbersome search through a myriad of systems as in legacy systems to identify what corrective actions are to be performed. The actionable information is delivered to the user rather than expecting the user to access each system separately, regardless of whether there are actions to be performed or not. For instance, the actionable information is delivered directly to the user or as a response to query from another electronic system (e.g., data collector 120 can send a request for output from analytic component 113, and receives an automatic response).

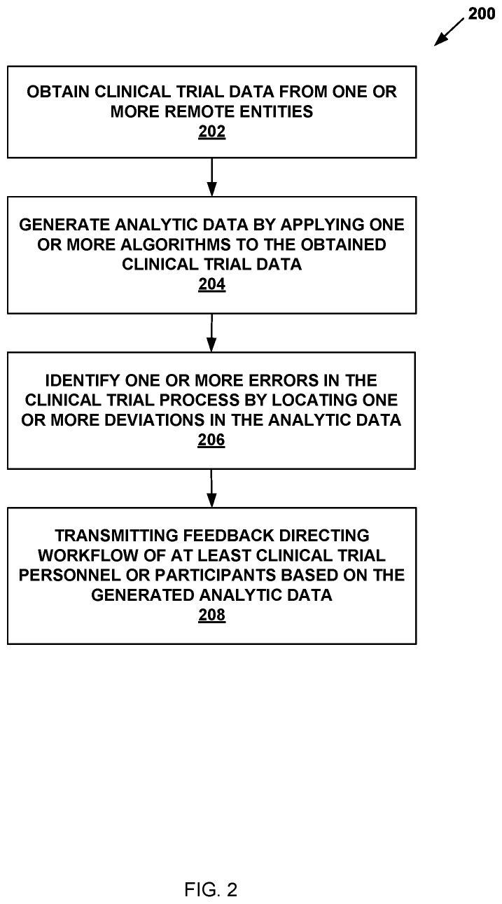

FIG. 2 illustrates an exemplary method 200 for identifying errors in clinical trial data and for directing workflow in a clinical trial process based on a specific clinical trial protocol and risk assessment performed by a clinical trial monitoring entity 102 according to the present disclosure. For instance, method 200 may include, at block 202, obtaining clinical trial data (e.g., clinical trial data 103) from one or more remote entities (e.g., remote entities 101). Furthermore, method 200 may include, at block 204, generating analytic data by applying one or more algorithms to the obtained clinical trial data. In some examples, these one or more algorithms can be designed specifically for the particular data within a particular trial. In addition, the method 200 can include, at block 206, identifying one or more errors in the clinical trial process by locating one or more deviations in the analytic data. Furthermore, method 200 may include, at block 208, transmitting feedback (e.g., feedback 105) directing workflow of at least some clinical trial personnel and/or participants based on the generated analytic data.

In addition, though not explicitly shown in FIG. 2, method 200 may include one or more additional or alternative embodiments, which are described above in reference to FIG. 1. For instance, method 200 can also include ensuring the directed workflow is completed according to a specific schedule. In addition, generating the analytic data at block 204 can include generating one or more unique reports (or data visualizations), trend analysis, etc. designed to evaluate the clinical trial data and direct workflow.

In some examples, identifying the one or more errors can include identifying which of the one or more remote entities 101 is a source of an error such that proper feedback for directing the workflow toward correction of the error is properly addressed. In addition, locating the one or more deviations in the analytic data can include obtaining control data (e.g., data for a particular clinical trial plan or schedule) for a particular report (or data visualizations) comprised of the analytic data. Additionally or alternatively, locating the one or more deviations in the analytical data could include obtaining expected or defined results for a particular report (or data visualizations) comprised of the analytic data. For instance, an expected result could be an action defined by a protocol (e.g., collecting an EKG measurement or blood sample). Locating a deviation could include locating analytical data indicating a research site did not collect an EKG measurement or a blood sample as outlined by a protocol at a certain research subject visit. As another example of an expected result, the protocol could have expectations for subjects (e.g., requirements for subjects or administering products to certain subjects). A deviation could be located if a site treats subjects with an investigational product when subjects do not meet the requirements of the protocol, or administer the wrong dose of an investigational product. Locating the one or more deviations includes comparing the analytic data to the control data and/or expected results, and identifying a deviation or trends of deviations where a difference between the analytic data and the control data and/or expected results meets a deviation criterion. This deviation criterion, for example, can be a threshold that can be preconfigured by a user, system operator, or manufacturer, or can be dynamically set to respond to certain parameter or environment changes present at a given time and/or for a given trial.

Furthermore, in some instances, the clinical trial data can be obtained from any device or technology used in the analysis of clinical trial data by importing the data over a communication line (e.g., clinical trial data including metadata associated with its collection), retrieving the data from memory, and/or receiving the data via manual entry. The obtained clinical trial data can also be verified by comparing the data to a larger set of comprehensive clinical trial data from a plurality of other remote entities.

The method 200 can also include presenting a notification to a user, where the notification can include an alert responsive to detecting a critical event indicator in the analytic data. Additionally or alternatively, the notification can be a periodic or customized notification associated with an action item to direct the workflow for the clinical trial. In some examples, the notification is presented to the user via text message, instant message, email, or other electronic communication technique, and can include one or more gaming components for mapping events associated with the clinical trial process. The method 200 can also include generating a comprehensive report of the performance of the clinical trial as a whole, one or more reviewers, and/or one or more reviewed entities. In addition, the analytic data, trend analysis, clinical trial data, and/or the report or reports (or data visualizations) can be forwarded to one or more data collectors. Furthermore, the clinical trial data can be monitored in real time or in near real time remotely such that any errors in the clinical trial process are identified in real time or near real time.

As a first non-limiting example of embodiments described herein (e.g., in accordance with the method 200 or system 10), obtained clinical trial data could comprise subject diary data (e.g., from an eDiary). Analytic data can be generated by applying one or more algorithms to data from the eDiary to generate information about the data from the eDiary. For instance a customized algorithm could evaluate the clinical trial data and generate analytic data indicating the number of days the subject completed the diary data, the average scores for certain assessments, the number of events that occurred within a specific time period, and whether the subject meets the criteria defined in the protocol. One or more errors could then be identified to determine whether a subject completing the diary meets criteria for enrollment (e.g., by comparing the analytic data to criteria for enrollment). The system then transmits feedback to the research site to confirm the subject is able to be enrolled or to identify the subject as not able to be enrolled in a study. For instance, the feedback directs clinical trial personnel to bar the subject from a clinical trial process or directs a clinical trial participant to participate in the study.

As a second non-limiting example of embodiments described herein (e.g., in accordance with the method 200 or system 10), obtained clinical trial data could include EDC data, metadata in an audit trail, training documentation of users to perform a particular assessment and/or clinical trial data indicating whether a delegation of authority grants permission to perform an assessment. One or more algorithms are used to extract the relevant clinical trial data from multiple databases and generate analytic data indicating whether the correct person has completed a critical assessment for the study as defined by a protocol. Deviations in the process for completing a critical assessment are determined from generated analytic data and feedback is transmitted. For instance, the feedback may direct a person to complete a critical assessment.

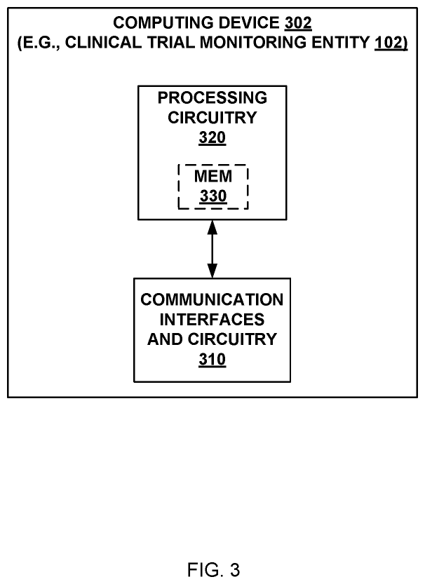

FIG. 3 illustrates additional details of an example computing device 302, which may be the clinical trial monitoring entity 102 of FIG. 1, in some examples, according to one or more embodiments. The computing device 302 is configured to implement processing to perform the aspects described above in reference to FIG. 2 and method 200. For instance, the computing device 302 is configured via functional components, means, or units (including but not limited to those components shown in clinical trial monitoring entity 202 in FIG. 1).

In at least some embodiments, the computing device 302 comprises one or more processing circuits 320 configured to implement processing of the method 200 of FIG. 2, such as by implementing functional means or units above. In one embodiment, for example, the processing circuit(s) 320 implements functional means or units as respective circuits. The circuits in this regard may comprise circuits dedicated to performing certain functional processing and/or one or more microprocessors in conjunction with memory 330. In embodiments that employ memory 330, which may comprise one or several types of memory such as read-only memory (ROM), random-access memory, cache memory, flash memory devices, optical storage devices, etc., the memory 330 stores program code that, when executed by the one or more for carrying out one or more microprocessors, carries out the techniques described herein.

In one or more embodiments, the computing device 302 also comprises one or more communication interfaces and circuitry 310. The one or more communication interfaces 310 include various components (e.g., antennas) for sending and receiving data and control signals. More particularly, the interface(s) 310 include a transmitter that is configured to use known signal processing techniques, typically according to one or more standards, and is configured to condition a signal for transmission (e.g., via a wired transmission line or over the air via one or more antennas). Similarly, the interface(s) include a receiver that is configured to convert signals received (e.g., via a modem or the antenna(s)) into digital samples for processing by the one or more processing circuits. The transmitter and/or receiver may also include one or more antennas or modems. By utilizing the communication interface(s) 310 and/or antenna(s), the computing device 302 is able to communicate with other devices to transmit feedback and receive data as well as manage the clinical trial processes as described above.

A computer program is also envisioned by the present disclosure, where the computer program comprises instructions which, when executed on at least one processor of the clinical trial monitoring entity 102 cause it to carry out any of the respective processing described above. Furthermore, the processing or functionality may be considered as being performed by a single instance or device or may be divided across a plurality of instances/devices of clinical trial monitoring entity 102 that may be present in a given system 10 such that together the device instances perform all disclosed functionality. Example embodiments further include a carrier containing such a computer program. This carrier may comprise one of an electronic signal, optical signal, radio signal, or computer readable storage medium. A computer program in this regard may comprise one or more code modules corresponding to the means or units described above.

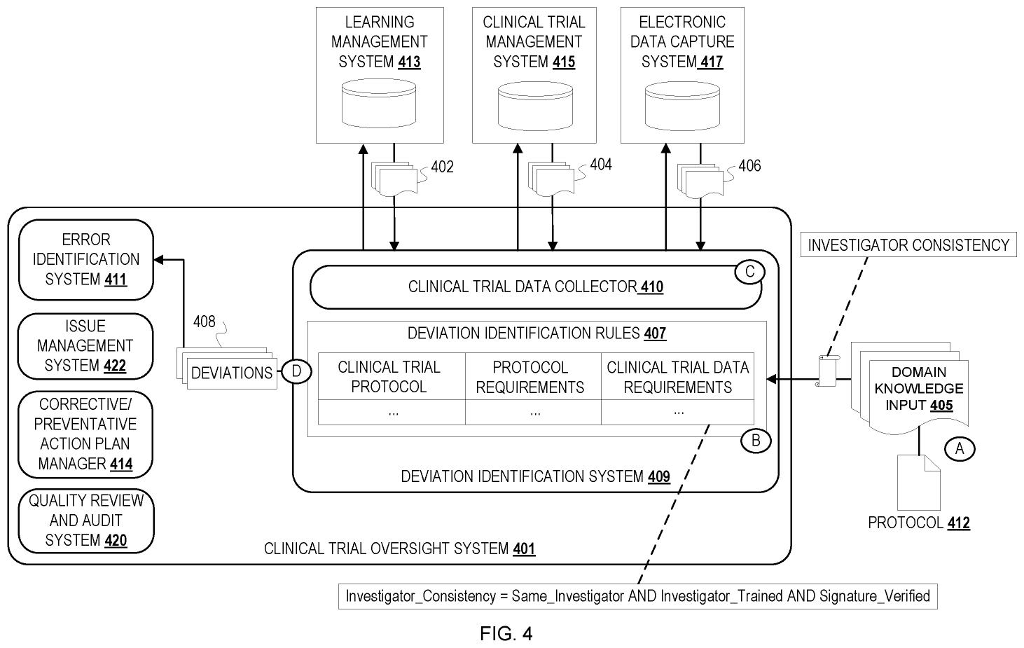

FIG. 4 depicts an example conceptual diagram of identifying deviations from a clinical trial protocol based on collected clinical trial data. A clinical trial oversight system 401 includes a deviation identification system 409, an error identification system 411, a CAPA manager 414, a quality review and audit system 420, and an issue management system 422. While depicted as part of the clinical trial oversight system 401, the deviation identification system 409, error identification system 411, CAPA manager 414, quality review and audit system 420, and/or issue management system 422 may be installed on different devices. A protocol 412 has been established for conducting a clinical trial. FIG. 4 also depicts three sources of clinical trial data which are accessible by the clinical trial oversight system 401: a learning management system 413 (e.g., a system which stores training data and/or documentation), a clinical trial management system 415, and an EDC system 417. Each of the learning management system 413, clinical trial management system 415, and EDC system 417 may be a server(s) which maintains a repository for the corresponding data which could be collected prior to or during the clinical trial. While the example depicted in FIG. 4 refers to collection of clinical trial data from a learning management system, a clinical trial management system, and an EDC system, clinical trial data can be collected from any system used for collection and storage of clinical trial data. For example, clinical trial data can be collected from an eConsent system alternatively or in addition to the sources of clinical trial data depicted in FIG. 4.

FIG. 4 is annotated with a series of letters A-D. These letters represent stages of operations. Although these stages are ordered for this example, the stages illustrate one example to aid in understanding this disclosure and should not be used to limit the claims. Subject matter falling within the scope of the claims can vary with respect to the order and some of the operations.

At stage A, domain knowledge input 405 is determined based on standards associated with the clinical trial and conducting the clinical trial, such as the protocol 412. For example, the domain knowledge input 405 may be determined by a clinical trial reviewer or principal investigator. Domain knowledge input 405 can be determined based on the protocol 412 prior to commencement of a clinical trial. During review of the protocol 412, aspects of conducting the clinical trial for which compliance with the protocol 412 is critical to maintain integrity of collected clinical trial data (“critical aspects”) are distinguished from “non-critical” aspects, or aspects which may not be directly related to or do not directly impact the integrity of data collected during the clinical trial. The domain knowledge input 405 indicates these critical aspects. For example, a critical aspect indicated in the domain knowledge input 405 may be an aspect of the protocol 412 related to informed consent documentation, where an aspect determined to be non-critical may be an aspect of the protocol 412 related to a publication policy. The domain knowledge input 405 can be determined based on other factors in addition to the protocol 412, such as clinical trial regulatory requirements, laws, a study plan associated with the clinical trial, etc. The domain knowledge input 405 can also be determined based on a medical condition which is being studied during the clinical trial. For instance, the domain knowledge input 405 can indicate expected progression of the medical condition and/or a rating of improvement of the medical condition (e.g., a benchmark(s) for determining improvement).

The domain knowledge input 405 also indicates aspects or conditions of the collected clinical trial data which are to be satisfied in order to comply with the critical aspects of the protocol 412 and/or other standards which are established for evaluation of processes for conducting the clinical trial. For instance, the domain knowledge input 405 may include expected values or ranges, baseline values, and/or controls corresponding to each identified critical aspect. The conditions for satisfying the critical aspects and corresponding expected values, baseline values, etc. can also be determined based on the protocol 412, regulatory requirements, laws, etc., or any combination thereof. As an example, if the domain knowledge input 405 is determined based on a medical condition being studied, the conditions can correspond to patterns of the medical condition such that clinical trial data can be evaluated to determine if the collected data are consistent with the expected patterns of the medical condition. The critical aspects to be monitored and the corresponding conditions for the clinical trial data indicated in the domain knowledge input 405 may be determined based on a primary efficacy endpoint selected for the clinical trial and/or a process for measuring the primary efficacy endpoint which is indicated in the protocol 412.

In this example, consistency in investigator attendance, duties, and training may be determined to be a critical aspect of conducting the clinical trial in accordance with the protocol 412. The domain knowledge input 405 thus indicates a critical aspect of “Investigator Consistency.” To satisfy this critical aspect of the protocol 412, the domain knowledge input 405 may indicate that the collected clinical trial data should reflect that the same investigator has been responsible, that the investigator has been trained appropriately, and that each signature field requesting the investigator's signature is completed with a verified signature.

At stage B, the deviation identification system 409 determines the sources from which to collect clinical trial data for analysis. The deviation identification system 409 determines the sources based on deviation identification rules (hereinafter “rules”) 407 which are attached to (e.g., installed on or otherwise accessible to) the deviation identification system 409. The rules 407 can be constructed based on the domain knowledge input 405 prior to commencement of the clinical trial. The rules 407 indicate rules for satisfying the critical aspects of conducting the clinical trial in compliance with the protocol 412 based on the domain knowledge input 405, such as to ensure that processes for conducting the clinical trial have been correctly followed. For instance, the rules 407 can indicate the critical aspects identified in the domain knowledge input 405 and the conditions for the clinical trial data to satisfy the critical aspects. Expected values or ranges, baseline values, and/or controls may be associated with the conditions indicated in the rules 407 (e.g., based on the domain knowledge input 405).

In this example, the rules 407 indicate a rule Investigator_Consistency which corresponds to the critical aspect of “Investigator Consistency” indicated in the domain knowledge input 405. The rules 407 indicate that the clinical trial data collected for a clinical trial participant (e.g., a research subject) should indicate that the same investigator was involved in completing the prescribed duties, the investigator was properly trained, and an electronic signature of the investigator was present and verified in order for the clinical trial data to satisfy the rule for investigator consistency. For instance, an expected value for the Same_Investigator condition may be an identifier (ID) assigned to an investigator responsible for overseeing one or more assessments completed at a study site as outlined in the protocol 412. An expected value for the Investigator_Trained condition may be an indication that the investigator's institutional review board (IRB) training is valid. An expected value for the Signature_Verified condition may be that the investigator's electronic signature was present and could be verified for documentation with an investigator signature field.

The deviation identification system 409 determines the sources of clinical trial data which is to be collected to evaluate the clinical trial data in view of the rules 407. In this example, the clinical trial data to be evaluated for compliance with the rules 407 is stored in the learning management system 413, the clinical trial management system 415, and the EDC system 417. The rules 407 may indicate sources of clinical trial data from which the clinical trial data to be analyzed are to be retrieved. For example, the rules 407 may also indicate that the clinical trial data are to be retrieved from the learning management system 413, the clinical trial management system 415, and the EDC system 417. Alternatively, another set of rules, policies, mappings of clinical trial data types to respective sources, etc. may be attached to the deviation identification system 409.

At stage C, a clinical trial data collector 410 collects the clinical trial data associated with the clinical trial data conditions which are indicated in the rules 407. Collection of clinical trial data can be tailored to the particular critical aspects of a protocol associated with a clinical trial (e.g., the protocol 412) to facilitate a focused assessment of clinical trial data. The clinical trial data collector 410 collects the clinical trial data from the sources of clinical trial data which the deviation identification system 409 determined at stage B. Collection of clinical trial data may be triggered based on the elapsed time since the previous data collection event exceeding a time threshold. For instance, the clinical trial oversight system 401 may enforce a time threshold of 24 hours such that the clinical trial data collector 410 collects clinical trial data for daily analysis. The clinical trial data collector 410 may collect clinical trial data for each enrolled participant across each study site, where the clinical trial data may be stored in databases or repositories maintained for different data collection systems. The clinical trial data collector 410 can collect the clinical trial data which was stored since a previous clinical trial data collection event (e.g., based on a time stamp associated with the clinical trial data).

In this example, the clinical trial data collector collects training data 402 from the learning management system 413, clinical trial management data 404 from the clinical trial management system 415, and EDC data 406 (e.g., electronic case report forms, audit trails, and metadata including electronic signatures) from the EDC system 417. The training data 402, clinical trial management data 404, and EDC data 406 are hereinafter collectively referred to as the “clinical trial data 402, 404, 406.” The clinical trial data 402, 404, 406 may be data which were collected directly for a participant during an assessment, data collected in association with a process for conducting the clinical trial, and/or data collected to document clinical trial procedure associated with the participant (e.g., operational data). For instance, the EDC data 406 may be electronic case report forms indicating participant medical data which an investigator has signed, whereas the clinical trial management data 404 may be data collected from the investigator, such as delegation of duties from a Principal Investigator. The clinical trial data 402, 404, 406 may also include associated metadata (e.g., audit trail data). For instance, training data 402 may include data documenting required training in addition to the audit trail associated with who completed and when the training was completed.

To collect the clinical trial data 402, 404, 406, the clinical trial data collector 410 can communicate a request to each of the learning management system 413, clinical trial management system 415, and EDC system 417. The deviation identification system 409 may first establish a channel for secure communication with the learning management system 413, clinical trial management system 415, and/or EDC system 417 before communicating the request, such as per the Secure Sockets Layer (SSL) or Transport Layer Security (TLS) protocol. The deviation identification system 409 may maintain a list which indicates a unique ID which has been defined for each participant and/or study site. The request may indicate one or more participant IDs and/or study site IDs associated with participants enrolled in the clinical trial. The participant IDs may also be leveraged for de-identification of the clinical trial data 402, 404, 406. The clinical trial data collector 410 can then obtain the training data 402 from the learning management system 413, clinical trial management data 404 from the clinical trial management system 415, and the EDC data 406 from the EDC system 417 for the participants enrolled at one or more study sites and the clinical trial staff associated with the trial as a whole or an individual study site.

At stage D, the deviation identification system 409 analyzes the clinical trial data 402, 404, 406 to identify deviations 408 based on the rules 407. The deviation identification system 409 analyzes the clinical trial data 402, 404, 406 collected for participants with respect to the rules 407 to determine if the clinical trial data 402, 404, 406 indicate a deviation from the protocol 412. The analysis of the clinical trial data 402, 404, 406 for deviation identification is thus customized to the critical aspects of the protocol 412 as indicated in the domain knowledge input 405. The deviation identification system 409 inspects particular data fields of the clinical trial data 402, 404, 406 collected for each participant based on the conditions in the rules 407. As an example, rather than analyzing each of the fields in the EDC data 406 (e.g., an electronic case report form), the deviation identification system 409 may inspect the EDC data 406 to determine if a signature corresponding to a certain authorized investigator is indicated in a signature field. The deviation identification system 409 can determine the data field to evaluate based on the rules 407.

The identified data fields of the clinical trial data 402, 404, 406 collected for each participant are evaluated against the rules 407 to determine whether the rules 407 are satisfied or whether a deviation from the protocol 412 has occurred. The deviation identification system 409 may enforce any criteria for identifying deviations. A state or history may be maintained for the rules 407 which is updated to include one or more values which the deviation identification system 409 has determined conform to the rules 407 during clinical trial data evaluation. The deviation identification system 409 can then evaluate a value of a data field of the clinical trial data 402, 404, 406 based on comparison with a corresponding value included in the state, history, etc. maintained for the rules 407. For instance, the deviation identification system 409 may determine an investigator ID which satisfies the rules 407 during analysis of the clinical trial data 402, 404, 406 and can then record the investigator ID in association with the rules 407. The deviation identification system 409 may identify deviations in the clinical trial data 402, 404, 406 based on a value of a particular data field failing to match the conditions indicated in the rules 407, such as based on the value failing to match an expected value or the value included in the state, history, etc. associated with the rules 407, falling outside of an expected range, etc. The deviation identification system 409 may also enforce a deviation threshold between a value of a data field and an expected value.

As an example, the deviation identification system 409 can analyze the clinical trial data 402, 404, 406 corresponding to a participant with participant ID 217 to determine if the same investigator has electronically signed electronic case report forms associated with participant ID 217, if the investigator performing assessments for the participant ID 217 was properly trained, and if the investigator's electronic signature could be verified for the documentation associated with the participant ID 217. Discrepancies in the investigator ID indicated in EDC data 406 collected for the participant ID 217 and the investigator ID indicated as an expected value in the rules 407, for example, would then trigger identification of the EDC data 406 collected for the participant with participant ID 217 as one of the deviations 408, or a deviation from the Same_Investigator condition. The deviation identification system 409 may associate a label with, tag, etc. the deviations 408 to indicate the type of deviation (e.g., a deviation from the Investigator_Trained condition). The deviations 408 may also indicate the participant ID, investigator ID, and/or study site ID and at least a subset of the clinical trial data associated with each deviation. The deviation identification system 409 may group the deviations 408 by study site ID, investigator ID, deviation type, etc.

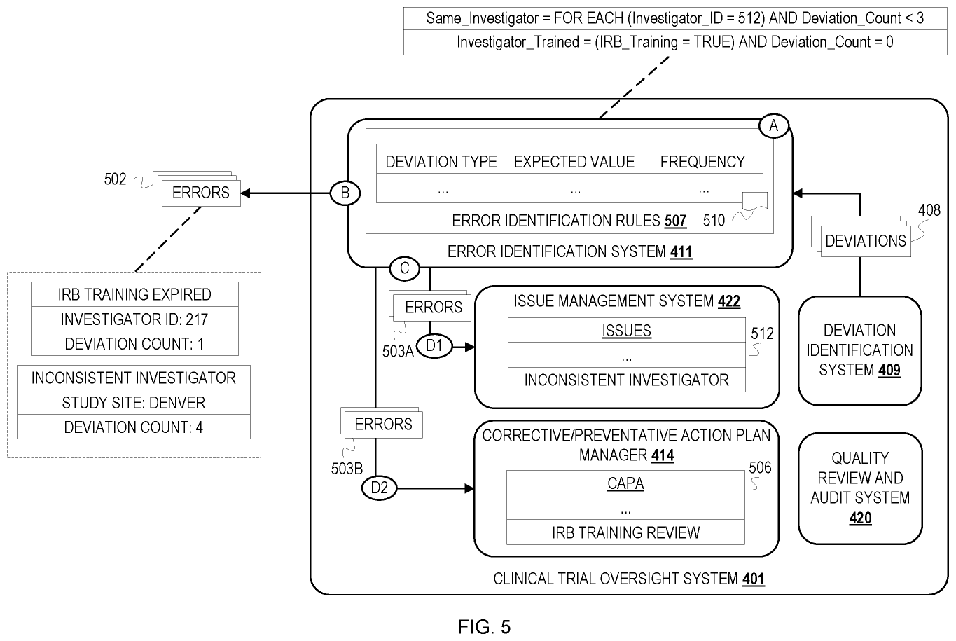

Once the deviations 408 have been identified, the deviation identification system 409 may indicate the identified deviations 408. For instance, the deviation identification system may generate notifications indicating the subset of the deviations 408 identified for each study site to be communicated to clinical trial personnel at the study sites. Alternatively or in addition, the deviation identification system 409 may store the deviations 408 in a central repository (e.g. a repository maintained by the clinical trial monitoring system or a repository maintained by a server which is accessible to the deviation identification system 409) from which clinical trial personnel can access information about the deviations 408. The deviation identification system 409 can also populate a form(s) (e.g., deviation forms, action item forms, etc.) with data and/or metadata of the deviations 408. The deviation identification system 409 communicates the deviations 408 to an error identification system 411 for further analysis to determine whether the deviations 408 correspond to systematic or high impact errors, outliers, or trends in deviations from the protocol 412. Analysis of the deviations 408 is further described in reference to FIG. 5.

Though not depicted in FIG. 4, the deviation identification system 409 may also maintain one or more rules for discontinuing the clinical trial. The deviation identification system 409 can evaluate clinical trial data, such as the clinical trial data 402, 404, 406, against the rule(s) for discontinuing the clinical trial as described in reference to evaluating the clinical trial data 402, 404, 406 against the rules 407. For instance, the rule(s) for discontinuing the clinical trial may indicate one or more criteria which, when satisfied by at least a subset of the clinical trial data 402, 404, 406, result in the clinical trial oversight system 401 indicating that the clinical trial should be discontinued (e.g., by generating a notification).

FIG. 5 depicts an example conceptual diagram of analyzing identified clinical trial protocol deviations to identify errors in conducting the clinical trial, such as systematic errors and/or high impact errors (collectively referred to herein as “errors”). As used herein, systematic errors and high impact errors refer to errors identified based on analysis of deviations from the clinical trial protocol, study plan associated with the clinical trial protocol, or another standard established for conducting the clinical trial which indicate instances of non-compliance with the protocol, study plan, or other standard. Systematic errors can be distinguished from outliers or high impact errors in that systematic errors repeat or occur multiple times. A few examples of a systematic error include a deviation or set of similar deviations that occurs multiple times across sites or at a same site, and a deviation or set of similar deviations that are associated with a same individual or study site(s). This repetition of a deviation(s) suggests or indicates an ongoing process issue. High impact errors may be considered lower-frequency errors which can be distinguished from outliers in that the high impact errors can affect integrity of clinical trial data and/or safety of clinical trial participants. Systematic errors and high impact errors can be identified based on evaluating the deviations 408 with respect to criteria for systematic error and high impact error identification which have been established for the clinical trial protocol. For instance, a type of deviation with a high frequency of occurrence may be considered a systematic error, while a type of deviation with a lower frequency of occurrence with serious implications for maintaining integrity of the clinical trial data may be considered a high impact error. FIG. 5 depicts the clinical trial oversight system 401 and the deviation identification system 409, error identification system 411, CAPA manager 414, quality review and audit system 420, and the issue management system 422 also depicted in FIG. 4. The operations depicted in FIG. 5 can be performed after the deviations 408 are passed from the deviation identification system 409 to the error identification system 411.

FIG. 5 is annotated with a series of letters A-D2. These letters represent stages of operations. Although these stages are ordered for this example, the stages illustrate one example to aid in understanding this disclosure and should not be used to limit the claims. Subject matter falling within the scope of the claims can vary with respect to the order and some of the operations.

At stage A, the error identification system 411 analyzes the deviations 408 based on error identification rules (“rules”) 507. The rules 507 can indicate criteria for determining that deviations from one or more protocol requirements, such as the protocol requirements included in the deviation identification rules 407 described in reference to FIG. 4, comprise a systematic error or high impact error. For instance, the criteria can include frequency thresholds for deviations from expected values of one or more data fields, values which were determined to satisfy the rules 407 indicated in a state maintained for the rules 407, etc. Criteria can be established for protocol requirements individually. As an example, the protocol requirements of Same_Investigator and Investigator_Trained can be indicated in the rules 507 with criteria for expected values and frequency thresholds. A state 510 is also maintained for the rules 507 which indicates a deviation count for each of the rules 507 that the error identification system 411 can update upon identifying an instance of a deviation. The error identification system 411 may then identify errors based on the counts of instances of deviations from Same_Investigator and Investigator_Trained maintained in the state 510 exceeding the frequency threshold. Determining that a subset of the deviations 408 with a same deviation type fails to satisfy the rules 507 can result in the error identification system 411 determining that the deviation type constitutes a systematic error or a high impact error.

Errors in conducting the clinical trial in accordance with the protocol can be identified for any level of granularity. For instance, errors can be identified for individual study team members, investigators, study sites, regions composed of multiple study sites, etc. In this example, the rules 507 indicate that for the Same_Investigator protocol requirement, an expected value of the investigator ID is a value of 512 with a frequency threshold of three (e.g., for a study site). In this case, identifying more than three occurrences at a study site in which an investigator ID did not match the expected investigator ID prompts a determination that investigator inconsistency is a systematic error in conducting the clinical trial at that study site. The rules 507 also indicate that for the Investigator_Trained protocol requirement, an investigator should have valid IRB training, where a deviation count greater than 0 results in identifying improper training of investigators as a high impact error.

The error identification system 411 may use any statistical method or combination of statistical methods for analyzing the deviations 408 based on the rules 507. For instance, the error identification system 411 may leverage the mean, standard deviation, variance, etc. and/or may conduct one or more statistical tests with the clinical trial data included in the deviations 408 for identifying outliers and/or trends in the clinical trial data underlying the deviations 408. The error identification system 411 can evaluate the results of the statistical analysis or analyses of the deviations 408 in light of the rules 507. Subsets of the deviations 408, either individually or grouped by deviation type, which do not comply with the rules 507 are identified as errors 502. The errors 502 may be high impact errors and/or systematic errors. The error identification system 411 may log the errors 502 which have been identified, where a log entry may include the error type, the entity or entities for which the deviation was identified (e.g., an investigator and/or study site ID), and/or a count of the deviations based on which the errors 502 were identified that is indicated in the state 510. The error identification system 411 can also isolate or generate a command to isolate the clinical trial data which were impacted by the identified errors 502. For instance, the error identification system 411 may communicate a notification to an entity which manages clinical trial data collected during the clinical trial which indicates the participant IDs, study site IDs, etc. and a time stamp associated with the clinical trial data for which the deviation was identified.

At stage B, the error identification system 411 indicates the errors 502 which have been identified. The error identification system 411 can, for instance, generate a report(s) and/or a notification(s) which indicates the identified errors 502. The report(s) and/or notification(s) may be communicated to the study site or study sites at which the errors 502 occurred. Alternatively or in addition, the error identification system 411 can associate a label with, tag, or otherwise identify the errors 502. The errors 502 may be grouped by error type, study site ID, investigator ID, etc. In this example, the errors 502 indicate that the compliance with the Investigator_Trained protocol requirement for the investigator with investigator ID 217 has been identified as a high impact error, with a deviation count of one, and that compliance with the Same_Investigator protocol requirement for the study site located in Denver has been identified as a systematic error, with a deviation count of four. The error identification system 411 can also guide the subsequent workflow in conducting the clinical trial based on identifying the errors 502 to address the errors 502. For instance, the error identification system can indicate the study site(s) for which the errors 502 were detected and/or the associated study team member(s) such that an onsite monitoring visit, training session, meeting with the principal investigator, and/or a meeting with the identified study team member(s) can be scheduled. As another example, the error identification system 411 can indicate one or more actions to perform to address the errors 502, such as a training item to complete to remediate the cause of one or more of the high impact and/or systematic errors.

At stage C, the error identification system 411 analyzes the errors 502 to determine the errors which are sufficiently consequential such that a CAPA should be developed to address those errors and prevent subsequent occurrences of those errors in conducting the clinical trial. Distinguishing errors for which a CAPA should be developed from other errors may be based on the error types and/or frequencies of error types. For instance, the error identification rules 507 may also indicate for each protocol requirement whether a CAPA should be developed if an error is identified. As another example, another set of rules or policies may be attached to the error identification system 411 which designate at least one criterion for logging an error for development of a CAPA. The error identification system 411 determines that a subset of the errors 502 should be logged as issues without development of a CAPA and a different subset of errors 502 should be logged for CAPA development, depicted as errors 503A and errors 503B, respectively.

At stage D1, the issue management system 422 logs the errors 503A as issues in an issue log 512. In this example, the error identification system 411 determines that the error in complying with the Same_Investigator protocol requirement does not satisfy the criterion for CAPA development and communicates the corresponding errors 503A to the issue management system 422. The issue management system 422 receives the errors 503A and records an entry in an issue log 512 based on the errors 503A. The issue log 512 can indicate the detected errors in conducting the clinical trial for subsequent analysis and evaluation of the impacted clinical trial data, clinical trial participants, etc.

At stage D2, the CAPA manager 414 logs the errors 503B for development of a CAPA. In this example, the error identification system 411 determines that a CAPA should be developed for the error in complying with the Investigator_Trained protocol requirement and communicates the corresponding errors 503B to the CAPA manager 414. The CAPA manager 414 receives the indication of the errors 503B and records an entry in a CAPA log 506 based on the errors 503B. The CAPA log 506 can indicate items for which a CAPA is to be developed and may also indicate CAPAs in progress (e.g., previously logged items for CAPA development). Clinical trial reviewers can thus review the CAPA log 506 to determine that a CAPA is to be developed to address the errors 503B. While FIG. 5 depicts communication of the errors 503A-B to the issue management system 422 and the CAPA manager 414 as sequential operations, the operations depicted at stages D1 and D2 may be performed concurrently.

Though not depicted in FIG. 5, a state which corresponds to observed protocol compliance may be maintained for one or more of the rules 507 in addition to the state 510. For instance, a counter can be maintained for each of the rules 507 which indicates a number of consecutive error analysis events in which the respective rule was satisfied. Values of the state may be leveraged to facilitate determination of how quickly clinical trial conduct has returned to compliance with the protocol after the errors 502 have been identified, such as after a CAPA indicated in the CAPA log 506 has been implemented. For instance, a compliance state maintained in association with each of the rules 507 may be updated with a value indicating a lack of compliance with the corresponding rule(s) after the errors 502 are identified, such as a value of zero. During a subsequent error analysis, if an error in complying with one or more of the rules 507 is not identified, the compliance state maintained for the respective one(s) of the rules 507 can be updated with a value indicating that the rule(s) was satisfied (e.g., by incrementing the counter).

The error identification system 411 can determine whether an error has been resolved and the clinical trial has returned to compliance with the rules 507 following each state update event. The error identification system 411 may, for instance, maintain a compliance threshold. The compliance threshold can indicate a number of consecutive error analysis events in which the rules 507 should be satisfied after initially identifying an error to determine that the clinical trial has returned to compliance with the protocol and that the error has been remediated, corrected, etc. The error identification system 411 can therefore determine that an error has been remediated based on a value of the state maintained for the corresponding one of the rules 507 satisfying the compliance threshold. As an example, a compliance threshold of three can be established for the Same_Investigator rule. After the state maintained for the Same_Investigator rule has been updated to indicate compliance with the rule for three consecutive error analysis events, the error identification system 411 can determine that the error of investigator inconsistency has been resolved. If the error identification system 411 identifies an error based on a failure to satisfy one or more of the rules 507 during a subsequent error analysis, the error identification system 411 can “reset” the state value (e.g., to a value of zero) to indicate that the clinical trial is no longer being conducted in compliance with the protocol or other standard for which the rules 507 are established.

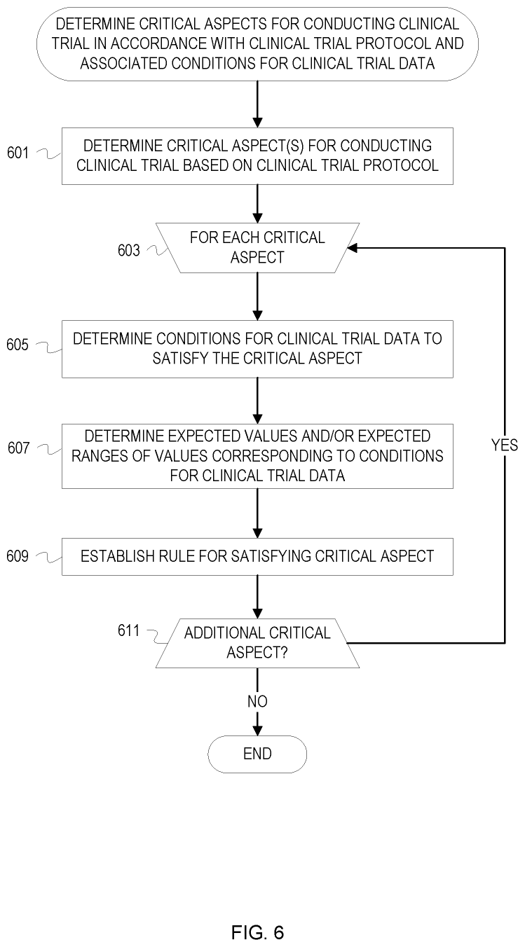

FIG. 6 is a flowchart of example operations for determining critical aspects for conducting a clinical trial in accordance with a clinical trial protocol. The example operations refer to a clinical trial reviewer as performing the depicted operations. As used herein, a clinical trial reviewer can be any entity which can generate domain knowledge input that can be leveraged for identifying protocol deviations, such as an entity which generates the domain knowledge input 405 described in reference to FIG. 4.

At block 601, the clinical trial reviewer determines one or more critical aspects for conducting a clinical trial based on a protocol which has been established for the clinical trial. The critical aspects of conducting a clinical trial can be distinguished from the non-critical aspects of conducting the clinical trial based on determining factors which, if not in compliance with the protocol, could jeopardize the integrity of the collected clinical trial data. The clinical trial reviewer may determine critical aspects based on other guidelines alternatively or in addition to the clinical trial protocol, such as regulatory requirements, laws, other clinical trial management documents, etc. As an example, with reference to FIG. 4, the clinical trial reviewer can determine the critical aspects for which rules are to be generated, such as the deviation identification rules 407, based on a review of the protocol 412. The critical aspects may be determined based on a primary efficacy endpoint which is established for the clinical trial (e.g., in the protocol). For instance, for a clinical trial for which mortality rate is a primary efficacy endpoint, the critical aspects may be determined to be that the same investigator completed the assessments for each assessment and that the investigator entered the data collected during the assessments at the same time of day. The clinical trial reviewer may determine the critical aspects based on other standards associated with the clinical trial alternatively or in addition to the protocol (e.g., study plans created for the clinical trial and/or expected progression of a medical condition being studied)

At block 603, the clinical trial reviewer begins reviewing each critical aspect. Each of the critical aspects determined at block 601 can be reviewed to establish a rule for satisfying the critical aspect.

At block 605, the clinical trial reviewer determines the conditions for clinical trial data to satisfy the critical aspect. The conditions may be considered the conditions for determining that a process for performing an assessment or otherwise conducting the clinical trial has been in compliance with the protocol based on analysis of the clinical trial data. Conditions for clinical trial data may be established to determine what aspects of clinical trial data should be inspected and may also indicate values or ranges of values are expected to be present in the clinical trial data to determine that the critical aspect has been satisfied. For example, for a critical aspect indicating that data collected during assessments should be entered at the same time of day, one condition may be that data collected when performing an assessment should be entered in an expected time window each time the assessment is performed. The conditions may correspond to conditions for clinical trial data related to study subjects, such as medical and process data, conditions for operational data (e.g., data collected for study sites), and/or associated metadata.

At block 607, the clinical trial reviewer determines expected values and/or expected ranges of values which correspond to the conditions for the clinical trial data. For instance, the clinical trial reviewer may determine expected values and/or ranges of expected values of certain data fields of clinical trial data associated with the conditions. As an example, for a condition that data collected for an assessment should be entered in an expected time window each time the assessment is performed, a window of expected times can be established against which time stamps of entered data can be compared, such as a window indicating that time stamps associated with data entered during an assessment should be between 10:30:00 AM and 11:15:00 AM.

At block 609, the clinical trial reviewer establishes a rule for satisfying the critical aspect. The rule can be established based on associating the critical aspect with the one or more conditions for satisfying the critical aspect. The rule, for instance, can indicate the critical aspect, the conditions for clinical trial data to satisfy the critical aspect (e.g., expected values and/or ranges of values), and data fields which should be evaluated against the conditions (e.g., a time stamp). The rule may also indicate a deviation criterion for identifying deviations from one or more of the conditions defined in the rule. The rule may be established as a programmatic rule. For instance, a deviation criterion may be a difference threshold, where a deviation is identified if the difference between the expected value and the actual value of a data field exceeds the difference threshold. A deviation criterion may also be an indication that a deviation should be identified if the actual and expected values of the data field do not match, if the actual value of the data field is not within a range of expected values, etc.

At block 611, the clinical trial reviewer determines whether there is an additional critical aspect for which a rule is to be constructed. If there is an additional critical aspect, operations continue at block 603. If there is not an additional critical aspect, operations are complete. Once the rules have been generated, the rules can be installed on or made available to a clinical trial oversight entity, such as the clinical trial oversight system 401 depicted in FIG. 4.

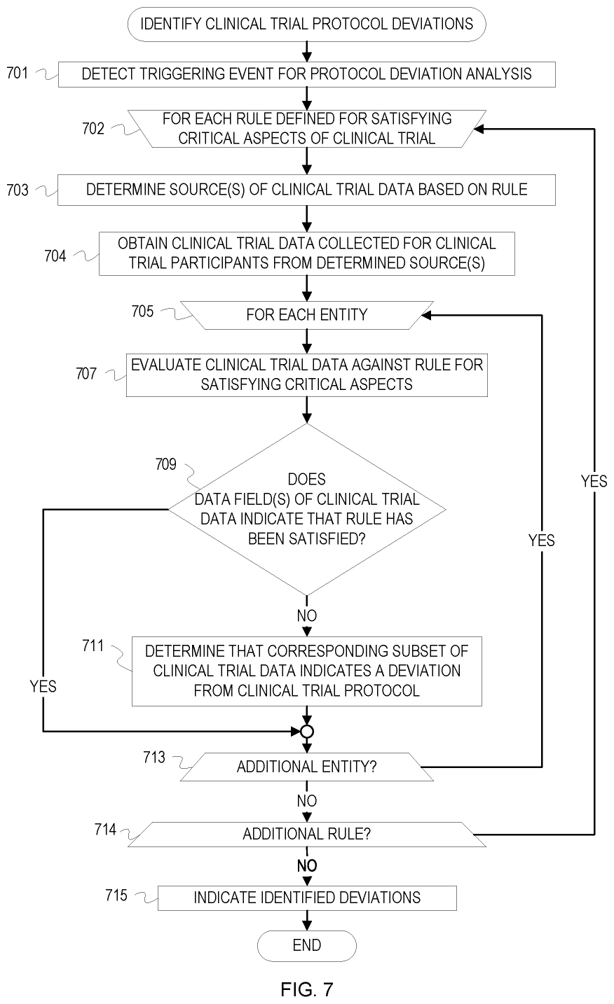

FIG. 7 is a flowchart of example operations for identifying deviations from a clinical trial protocol based on analysis of collected clinical trial data. The example operations refer to a deviation identification system as performing the example operations for consistency with FIG. 4, although naming of software and program code can vary among implementations. The deviation identification system can identify deviations from a clinical trial protocol based on a set of rules for clinical trial protocol compliance, such as the rules generated with the operations described in reference to FIG. 6. This data analysis to detect deviations is ongoing through the clinical trial. A deviation data analysis can be triggered at predefined time intervals that may or may not be the same over the duration of the clinical trial. The system can also be programmed to evaluate site digital calendars/schedules defined for different sites of a clinical trial to determine when collections/assessments are performed and analyze for deviations accordingly (e.g., after every n collections/assessments). The system can also be programmed to monitor data sources to which the system is “connected.” The system can be configured to subscribe to various data sources and detect when x records have been edited and/or added to any one of the data sources. Configuration of when to trigger deviation analysis can vary (e.g., x records updated at a specified ratio of the connected data sources and/or when a specific data source is updated). The subscription to data sources can be with a publisher/subscriber system depending upon implementation of the data sources. A data source may present data in a format that can be parsed/processed by the deviation identification system. A data source may communicate to the deviation identification system format (e.g., fields, field attributes, field locations, etc.) of the data.

At block 701, the deviation identification system detects a triggering event for protocol deviation analysis. Triggers for protocol deviation analysis can be related to expiration of time interval, creation and/or updates of records which are stored in a clinical trial data source, etc. For instance, the deviation identification system can begin protocol deviation analysis based on determining that a predetermined time interval since a preceding protocol deviation analysis event has expired (e.g., an interval of 24 hours). As another example, protocol deviation analysis may be triggered based on determining that a threshold number of records has been updated, modified, or added to a clinical trial data source. The triggering event may trigger the protocol deviation analysis for one or more study sites, clinical trial management teams, or study team members based on the triggering event type. For example, the protocol deviation analysis can be triggered for each of a subset of study sites which form a region based on a schedule for protocol deviation analysis established for that region. The protocol deviation analysis can then be performed on clinical trial data collected for each of the study sites in the region.

At block 702, the deviation identification system begins evaluation of critical trial data for each rule defined for the clinical trial based on the protocol. One or more rules which have been defined for ensuring compliance with the protocol may be attached to the deviation identification system. Each of the rules can indicate critical aspects which were identified based on the protocol and conditions for satisfying the critical aspects, such as expected values of certain data fields of clinical trial data. The rule against which clinical trial data is being evaluated is hereinafter referred to as “the rule.” The rule can indicate critical aspects corresponding to clinical trial participants and/or critical aspects corresponding to operational aspects (e.g., aspects unrelated to the participants) of the clinical trial. A state or history may be maintained for the rule which is updated to include a value which conforms to the rule during the evaluation of clinical trial data. For instance, a state may be maintained which indicates a value of the clinical trial data which the deviation identification system determined to conform to the rule previously during the analysis. The value(s) included in the state or history may be initialized with an initial state or initial value before evaluation has begun (e.g., a null value).

At block 703, the deviation identification system determines one or more sources of clinical trial data based on the rule. The deviation identification system may determine the sources of clinical trial data based on the data fields indicated in the rules (e.g., an EDC system, an eDiary system, etc.). For example, the deviation identification system can determine that a data field to be evaluated based on the rule corresponds to a type of data stored in an EDC system. The deviation identification system may make this determination based on another set of rules, policies, etc. attached to the deviation identification system which indicate associations or mappings between data types and the clinical trial data sources which stores the data types. The sources of data can be sources which store data related to clinical trial participants, such as medical data or process data, and/or operational data.

At block 704, the deviation identification system obtains clinical trial data from the determined sources. The deviation identification system may first establish a secure communication channel with each of the sources (e.g., a connection secured with SSL/TLS). The deviation identification system can generate a request for data to communicate to each of the sources. The request may indicate the participant IDs of participants enrolled in the study, study site IDs of study sites at which the clinical trial is conducted, etc. For instance, the request may indicate that data are to be obtained for investigator IDs, study site IDs, and/or IDs for participants or any other personnel at a study site. A data source may have an application programming interface published to at least the deviation identification system to allow the deviation identification system to request data from the data source and monitor the data source for updates. For privacy and confidentiality, the deviation identification system may be given permissions to access the data source or the data sources may be configured to publish updates to the deviation identification system.

At block 705, the deviation identification system begins evaluation of clinical trial data for each entity for which clinical trial data was collected. The clinical trial data which was collected may be arranged based on the ID(s) associated with the request for the clinical trial data (e.g., arranged by study site ID, study team member ID, and/or participant ID). Since the source(s) of the clinical trial data was determined based on the rule being applied for the clinical trial, then the entity is effectively dictated by the rule also. The entity may be a participant, a study site for which study site data was obtained, clinical trial personnel (e.g., a clinical trial management team), an investigator, or any other individual involved in conducting the clinical trial. The clinical trial data obtained for evaluation against the rule may include the clinical trial data collected for or related to a participant as well as the clinical trial data collected for the study sites, investigators, etc. (e.g., operational data).

At block 707, the deviation identification system evaluates clinical trial data obtained for the entity against the rule. The deviation identification system can evaluate the clinical trial data against each of the conditions indicated in the rule which were defined for the critical aspect. The deviation identification system compares the data field(s) of the clinical trial data against the condition(s) defined in the rule to determine if values of the data fields satisfy the conditions. For instance, the deviation identification system can identify a data field in the clinical trial data which corresponds to the condition to compare the value of the data field with the expected value or range corresponding to the condition. The deviation identification system can determine whether the data field(s) contains a value(s) which satisfies the condition as a result of the comparison. The deviation identification system may also compare the value of the data field with a value(s) included in the state, history, etc. associated with the rule.

At block 709, the deviation identification system determines whether values of the data fields which were evaluated indicate that the rule has been satisfied. The deviation identification system may maintain deviation criteria for determining whether a deviation can be identified based on the value or values. For instance, the deviation criteria may indicate that the deviation identification system is to identify a deviation if a value of a data field does not match an expected value or a value included in a state of conformance associated with the rule, if a value of a data field is not within an expected range of values, if a value of a data field exceeds a threshold difference from a baseline value indicated in the rules, etc. If the values of the data fields which were evaluated do not indicate that the rule has been satisfied, operations continue at block 711. If the values of the data fields which were evaluated indicate that the rule has been satisfied, operations continue at block 713.

At block 711, the deviation identification system determines that at least a subset of the clinical trial data indicates a deviation from the protocol. The deviation identification system may log the identified deviation in a deviation log. For instance, the deviation log entry can include the entity ID (e.g., a participant ID, study site ID, investigator ID, etc.), a deviation type, and the expected and actual values of the data field for which the deviation was identified. The deviation identification system may mark (e.g., associate a label, tag, or identify) the clinical trial data as comprising a deviation. For instance, the deviation identification system may associate a label with the value of the data field corresponding to the deviation, the subset of clinical trial data in which the deviation was identified (e.g., an electronic case report form), or the clinical trial data collected for the entity. The clinical trial data for the entity which were marked may subsequently be isolated from the collected clinical trial data upon determining that the deviation is a result of a systematic error rather than a low-frequency outlier.

At block 713, the deviation identification system determines whether additional entities in the clinical trial remain. If additional entities remain, operations continue at block 705. If no additional entities remain, operations continue at block 714.

At block 714, the deviation identification system determines whether additional rules against which to evaluate clinical trial data remain. If additional rules remain, operations continue at block 701, where the deviation identification system selects the next rule for evaluation. If no additional rules remain, operations continue at block 715.

At block 715, the deviation identification system indicates the identified deviations. The deviation identification system may generate a report indicating the deviations included in the deviation log. As another example, the deviation identification system may generate a notification which is communicated to the study site(s) at which one or more deviations were identified. The deviation identification system may also populate a form with information about the deviation system. For instance, the deviation identification system may populate a deviation form which indicates a study site ID, deviation type, study site personnel ID(s) associated with the deviation, etc.

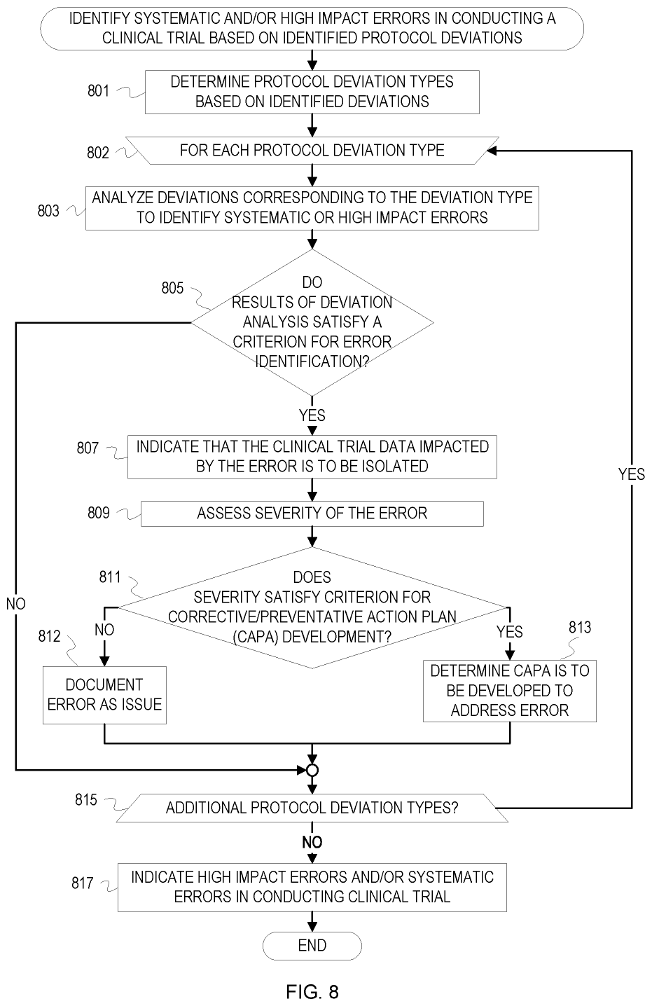

FIG. 8 is a flowchart of example operations for identifying high impact and systematic errors in conducting a clinical trial based on identified protocol deviations. Systematic errors and/or high impact errors in compliance with a clinical trial protocol when conducting the clinical trial can be identified based on evaluation of protocol deviations. The example operations refer to an error identification system as performing the example operations for consistency with FIG. 5, although naming of software and program code can vary among implementations. The example operations occur after protocol deviations have been identified based on analysis of clinical trial data. While the example operations refer to evaluation of deviations from a clinical trial protocol, the example operations may be performed based on evaluation of deviations from any standard which is established for conducting the clinical trial, such as a study plan, regulations, laws, and/or expected progression of a medical condition being studied.

At block 801, the error identification system determines one or more protocol deviation types based on the identified deviations. Protocol deviation types can be identified based on a critical aspect or condition for satisfying a critical aspect for which the deviation was detected. The error identification system can determine types of the identified deviations based on a label, tag, identifier, etc. which was associated with the deviation upon identification. For instance, with reference to FIG. 4, deviations corresponding to the Same_Investigator condition can be grouped as belonging to the same deviation type based on a label which indicates that the deviations were identified as deviations from the Same_Investigator condition for satisfying the critical aspect of Investigator_Consistency. As another example, entries in a deviation log accessible to the error identification system may include a field for deviation type for each logged deviation.

At block 802, the error identification system begins evaluation of deviations for each of the protocol deviation types. The error identification system can evaluate the subset of deviations which have been grouped based on type. For instance, the error identification system may evaluate deviations which are classified as deviations from the Same_Investigator condition for determining whether the deviations can be further classified as indicative of a systematic error.

At block 803, the error identification system analyzes the deviations corresponding to the deviation type to identify high impact errors or systematic errors in the deviations. Identification of a systematic error can involve maintaining a history of deviations that trigger a systematic error rule. A rule for identifying a systematic error specifies a standard (e.g., from a protocol or regulation) and a threshold number of deviations from that standard that would qualify as a systematic error. For example, a systematic error rule can be defined for fluid sample collection. The example rule can be defined with an acceptable time interval for fluid sample collections and deviation occurrence criteria. The system may initially evaluate past data across data sources against the rule to determine whether there have been n occurrences of deviations from the time interval for fluid sample collection specified as standard. As each occurrence of non-compliance is detected (i.e., the deviation criterion is satisfied), state data for the rule is updated (i.e., a counter is incremented). After state data is updated, the system evaluates the updated state data against the occurrence criterion to determine whether the deviation has occurred n times. After this initial evaluation, the state data for the systematic error rule is maintained across future evaluations. Embodiments may reset the state data for the systematic error rule when triggered (i.e., after identifying a repeated non-compliance as a systematic error). The error identification system can use statistical analysis to determine whether the deviations show a trend in protocol deviations which is indicative of a systematic error and/or whether any outliers are due to a high impact error. The error identification system may leverage a control group for statistical analysis which comprises control values for the data fields associated with the clinical trial data for which the deviation was identified. For example, the error identification system may leverage a control group for performing an unpaired t-test.

At block 805, the error identification system determines whether the results of the statistical analysis satisfy one or more criteria for high impact error identification or systematic error identification. The error identification system can enforce criteria for systematic error identification and high impact error identification based on results of analysis of the identified deviations to determine whether the results indicate the presence of a high impact or systematic error in conducting the clinical trial. A criterion for classifying an error as high impact or systematic may be established based on the protocol deviation type. Criteria for high impact or systematic error identification may include a threshold number of deviations or outliers, a determination that the results of the statistical analysis exhibit statistical significance based on a p-value not exceeding a significance level (e.g., p<0.05), etc. If the results of the statistical analysis satisfy one or more criteria for high impact or systematic error identification, operations continue at block 807. If the results of the statistical analysis do not satisfy one or more criteria for high impact or systematic error identification, operations continue at block 815.

At block 807, the error identification system indicates that the clinical trial data impacted by the high impact or systematic error is to be isolated. The error identification system can either generate notifications to cause isolation or perform operations to isolate the clinical trial data deemed as a indicating a high impact error or a systematic error. For instance, the error identification system can generate a notification or command which indicates the entity IDs (e.g., participant IDs, investigator IDs, etc.) and data fields associated with the identified error. The error identification system can communicate the notification to isolate the data to an entity which manages clinical trial data collected during the clinical trial. The impacted clinical trial data can then be removed from consideration (e.g., scrubbed) and/or training or other remediation can be undertaken to prevent further errors. As an example of isolating the clinical trial data, the error identification system can create a data object or populate a repository with the clinical trial data exhibiting a systematic error and identify or index by the corresponding entity ID. If the error identification system has permission to annotate or mark clinical trial data, the error identification system can set an isolator marker or tag to distinguish the clinical trial data as corresponding to a high impact error or a systematic error.

At block 809, the error identification system assesses the severity of the type of high impact or systematic error. The error identification system may maintain rules, policies, etc. for severity assessment of high impact errors and/or systematic errors which indicate error type and a frequency threshold, for example. For instance, a rule may be established which indicates that a systematic error in ensuring that the same investigator completes assessments is to be considered sufficiently severe if a frequency of deviations from this condition exceeds a threshold. Referring to FIG. 5, a systematic error corresponding to deviations from the Same_Investigator condition may be considered to sufficiently severe if the deviation count exceeds a threshold of three. As another example, another rule may be established which indicates that a high impact error of ensuring an investigator was properly trained is to be considered sufficiently severe if a count of deviations from this condition exceeds a threshold of one.

At block 811, the error identification system determines whether the severity of the protocol deviation satisfies a criterion for CAPA development. For instance, the error identification system can determine whether the frequency of deviations corresponding to the error exceeds the frequency threshold established for the error type and can be considered to be sufficiently severe. If the error does not satisfy a criterion for CAPA development, operations continue at block 812. If the error satisfies a criterion for CAPA development, operations continue at block 813.

At block 812, the error identification system documents the error as an issue. High impact errors or systematic errors which were not considered to be sufficiently severe for CAPA development may be recorded as an issue for subsequent evaluation of the clinical trial participants and/or clinical trial data impacted by the issue, investigation into the issue, etc. For instance, the error identification system may generate a notification or command to log the error in a central repository, log, etc. for documenting issues, such as an issue management system described in reference to FIG. 5.

At block 813, the error identification system determines that a CAPA is to be developed to address the high impact or systematic error. The error identification system may generate a notification or command to log the error in a centralized CAPA manager, such as a CAPA manager as described in reference to FIG. 5. The CAPA log can subsequently be accessed for development of a CAPA to address the error in conducting the clinical trial and the protocol deviations which resulted from the error.

At block 815, the error identification system determines if additional protocol deviation types have been identified. For instance, the error identification system can determine that additional deviation types have been identified if a deviation log indicated additional deviation types which were grouped at block 801. If additional protocol deviation types have been identified, operations continue at block 802. If additional protocol deviation types have not been identified, operations continue at block 817.

At block 817, the error identification system indicates the identified high impact errors and/or systematic errors. The error identification system can generate a report, a notification, etc. which indicates the errors which were identified. The error identification system may, for instance, group the errors by study site ID, investigator or other study team member ID, etc. The error identification system may also distinguish the errors which were logged for CAPA development. The indication of the errors (e.g., the report or notification) may indicate one or more corresponding actions and/or assignments to address or remediate the identified high impact and/or systematic errors to facilitate subsequent determination of when or whether the errors have been resolved. A listing of errors and the associated entities may also be stored or logged, such as in a repository maintained by the error identification system or a clinical trial oversight system on which the error identification system executes, thus providing a central resource for accessing information about high impact and systematic errors regardless of the systems in which the clinical trial data leveraged for the deviation and error analysis were initially stored.

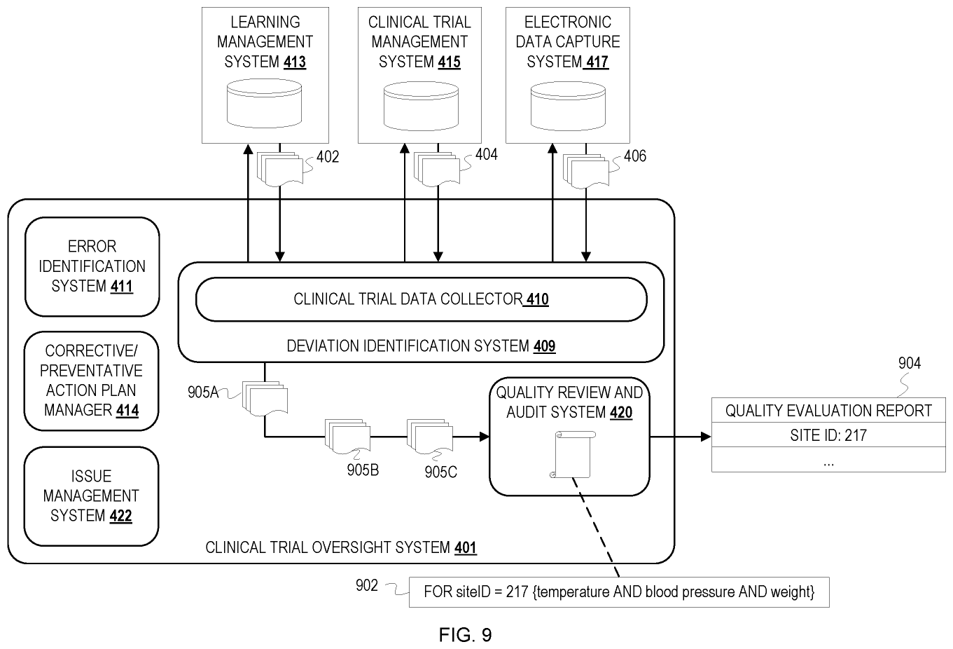

FIG. 9 is an example conceptual diagram of performing quality evaluation for an entity involved in a clinical trial based on collected clinical trial data. FIG. 9 depicts the clinical trial oversight system 401, deviation identification system 409, error identification system 411, CAPA manager 414, and quality review and audit system 420 also described in reference to FIGS. 4 and 5. The operations depicted in FIG. 9 can be performed concurrently with identification of deviations based on analysis of clinical trial data. Quality evaluation may be performed upon request or may be performed periodically based on a triggering event. For example, a quality evaluation of a study site may be performed after at least one systematic error or high impact error was detected for the study site and/or daily after a CAPA has been implemented at the study site. As another example, the system can trigger quality evaluations based on scheduled events of the clinical trial, such as monthly assessments of participants. For instance, quality evaluation can be triggered every mid-month based on a schedule of assessments or collections (data, sample collection, and/or measurements) at the beginning (e.g., first two weeks) of every month. The quality evaluation system can also trigger quality evaluations or adjust scheduled quality evaluations based on clinical trial calendars to accommodate changes (e.g., holidays, a rescheduled collection appointment, etc.).

The quality review and audit system 420 reviews a subset of the clinical trial data 402, 404, 406 which corresponds to the entity for which the quality evaluation is to be performed. The entity may be, for example, a monitor, data manager, investigator, a study site, or a region which includes more than one study site. The ID(s) of the entity being evaluated may be included in a request to perform the quality evaluation (e.g., based on input into a user interface). In this example, the quality review and audit system 420 reviews a randomly selected subset of the clinical trial data 402, 404, 406 which was collected at a study site with study site ID 217 based on study site IDs by which the clinical trial data 402, 404, 406 were arranged. The subset of the clinical trial data 402, 404, 406 which the quality review and audit system 420 evaluates, depicted in FIG. 9 as clinical trial data 905A-C, can be randomly selected based on a predetermined fraction, percentage, etc. of clinical trial data which the quality review and audit system 420 is to obtain for review (e.g., 5%, 10%, etc.). The clinical trial data 905A-C may be a copy of the corresponding clinical trial data 402, 404, 406 collected by the clinical trial data collector 410 which are already queued for deviation identification.

The quality review and audit system 420 determines the data fields of the clinical trial data 905A-C to evaluate based on monitoring criteria 902. The monitoring criteria 902 can indicate aspects or data points which are to be consistently monitored, documented, and/or reported during a clinical trial, such as data which should be collected during assessments performed for participants or data which should be submitted to the IRB. The monitoring criteria 902 may be determined based on a primary efficacy endpoint established for the clinical trial, for example. The monitoring criteria 902 may be different from the critical aspects identified based on the protocol or may overlap. In this example, the monitoring criteria 902 indicate that for each participant enrolled in the clinical trial at the study site with study site ID 217, the clinical trial data should reflect that temperature, blood pressure, and weight are measured during each assessment which is completed for the participant. Though the monitoring criteria depicted in this example refer to conditions for medical data, monitoring criteria can be established for any category of clinical trial data as well as any number of categories of clinical trial data. For instance, the monitoring criteria 902 could alternatively or additionally indicate conditions for audit trail data.

The quality review and audit system 420 evaluates the clinical trial data 905A-C to determine whether the monitoring criteria 902 have been satisfied. The quality review and audit system 420 can determine the data fields to be evaluated based on the monitoring criteria 902, such as similarly described in reference to deviation identification in FIG. 4. For instance, the monitoring criteria 902 can indicate the data fields which are to be evaluated. As another example, a set of policies or mappings of clinical trial data types to data points indicated in monitoring criteria 902 may be accessible to the quality review and audit system 420. The quality review and audit system 420 compares the respective data fields of the clinical trial data 905A-C to the monitoring criteria 902 to determine if values of the data fields indicate that the data points included in the monitoring criteria 902 have been documented or whether the clinical trial data 905A-C exhibit a deficiency with respect to the monitoring criteria 902.

Based on determining that the monitoring criteria 902 were not satisfied, the quality review and audit system 420 logs or records deficiencies in the clinical trial data 905A-C. For instance, the quality review and audit system 420 may log IDs associated with clinical trial participants or reports submitted to the IRB for which the monitoring criteria 902 were not satisfied. For example, the quality review and audit system 420 may log participant IDs and the associated subset of the clinical trial data 905A-C for which one or more of the temperature, blood pressure, and weight were not measured during an assessment. The quality review and audit system 420 can then produce a quality evaluation report 904 which indicates a quality review of the entity under evaluation based on the identified deficiencies. In this example, the quality evaluation report 904 may indicate a total fraction or percentage of participants enrolled at the study site with study site ID 217 for which the monitoring criteria 902 were not satisfied and a deficiency was thus identified, the investigators or individuals performing the assessments for which the deficiency was identified, etc. The quality review and audit system 420 may also determine an action(s) to be performed to address one or more of the identified deficiencies to be included in the quality evaluation report 904. For instance, the quality review and audit system 420 may determine a training item to be performed to address the identified deficiencies and to prevent repeated occurrences of the deficiencies (e.g., based on accessing a training database), a meeting with a study team member to be scheduled, an onsite monitoring visit to be scheduled, etc. The quality review and audit system 420 can then include the training item in the quality evaluation report 904.

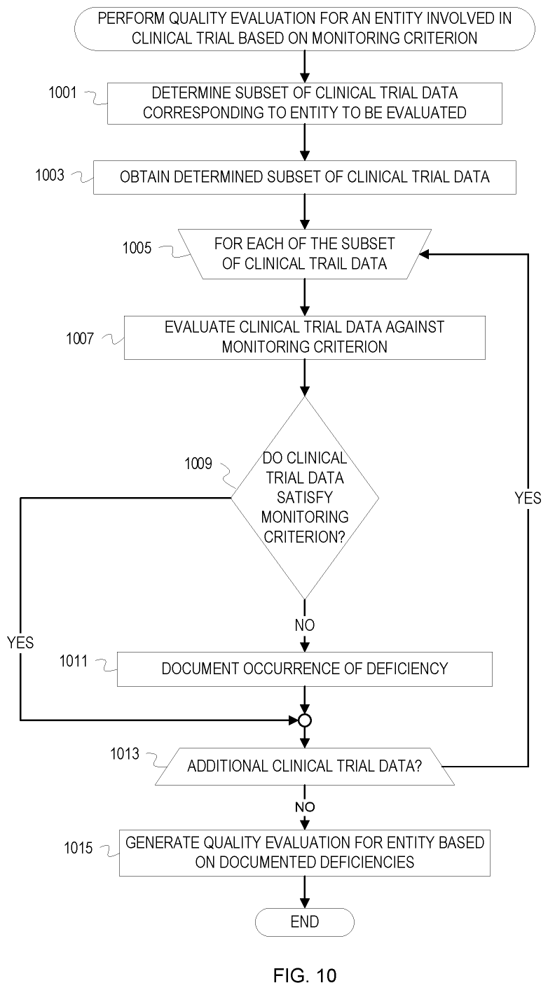

FIG. 10 is a flowchart of example operations for performing a quality evaluation of an entity involved in a clinical trial based on one or more monitoring criteria established for the clinical trial. The entity being evaluated may be, for example, a study team member (e.g., a monitor, data manager, a supervisor, or investigator/assessor), a study site, or a region formed by a collection of study sites. The example operations refer to a quality review and audit system as performing the example operations for consistency with FIG. 9, although naming of software and program code can vary among implementations. The operations depicted in FIG. 10 may be performed based on receiving a request for quality evaluation of an entity or may be performed periodically based on a triggering event (e.g., based on a schedule for quality evaluation assessments).

At block 1001, the quality review and audit system determines a subset of clinical trial data corresponding to the entity to be evaluated. For instance, if data collected for clinical trial participants is to be evaluated, a mapping of participant IDs to investigator IDs and/or study site IDs may be accessed by the quality review and audit system to determine the participants for which clinical trial data is to be evaluated. The quality review and audit system can randomly select a subset of the clinical trial data associated with the study team member ID(s), study site ID(s), etc. which corresponds to the entity being evaluated. The quantity of clinical trial data which the quality review and audit system randomly selects for evaluation may be based on a percentage or fraction. For example, the quality review and audit system may randomly select 5% of participants, reports, etc. associated with the entity being monitored.

At block 1003, the quality review and audit system obtains the subset of clinical trial data based on the random selection. The quality review and audit system may obtain the clinical trial data which has already been collected from clinical trial data sources, arranged based on ID(s) (e.g., participant ID, study team member ID, and/or study site ID), and queued for deviation analysis. For instance, as depicted in FIG. 9, the quality review and audit system may retrieve a copy of clinical trial data for the randomly selected subset of clinical trial data from a queue of clinical trial data, where the clinical trial data have been queued for a deviation analysis. As an example, if the quality evaluation is being performed for an investigator, the quality review and audit system can clinical trial data from the queue which was collected for 10% of clinical trial participants under the supervision of the investigator.

At block 1005, the quality review and audit system begins evaluating the subset of collected clinical trial data for each of the entities or items for which clinical trial data is included in the subset. For example, the subset of clinical trial data may include clinical trial data collected for participants or clinical trial data included in IRB reports. The quality review and audit system thus evaluates the clinical trial data collected for each participant or included in each IRB report.

At block 1007, the quality review and audit system evaluates the clinical trial data against at least one monitoring criterion. The quality review and audit system can enforce a monitoring criterion which indicates at least one data point which should be reviewed, documented, entered, etc. during an assessment. For example, the monitoring criterion can be a list of medical data which are to be collected during an assessment. The quality review and audit system determines the data field(s) which correspond to the monitoring criterion, such as based on an indication of the data field included in the monitoring criterion, a set of rules or mappings of monitoring criterion types to clinical trial data types, etc. The quality review and audit system can evaluate the clinical trial data to determine if the data field(s) indicate that the monitoring criterion has been satisfied.

At block 1009, the quality review and audit system determines whether the clinical trial data satisfy the monitoring criterion. Identification that a data point indicated in the monitoring criterion is missing from or incorrect in the corresponding data field in the clinical trial data can prompt a determination that the clinical trial data fails to satisfy the monitoring criterion. If the clinical trial data do not satisfy the monitoring criterion, operations continue at block 1011. If the clinical trial data satisfy the monitoring criterion, operations continue at block 1013.

At block 1011, the quality review and audit system documents the occurrence of a deficiency in the clinical trial data based on the monitoring criterion. For instance, the deficiency can correspond to a null value recorded for a data point indicated in the monitoring criterion or an incorrect value recorded for a data point indicated in the monitoring criterion, such as the absence of a measurement for a participant's blood pressure. The quality review and audit system may document the deficiency, ID of the affected entity or entities (e.g., a participant, IRB report, etc.), the expected value or range of values of the data field exhibiting the deficiency, and/or the actual value of the data field, such as by creating a new entry in a deficiency log maintained by the quality review and audit system.

At block 1013, the quality review and audit system determines whether additional clinical trial data of the subset of clinical trial data remain for quality evaluation. If additional clinical trial data remain, operations continue at block 1005. If no additional clinical trial data remain, operations continue at block 1015.

At block 1015, the quality review and audit system generates a quality evaluation for the selected entity based on the documented deficiencies. The quality evaluation for the entity may indicate a total quantity, percentage, etc. of the subset of clinical trial data for which a deficiency was identified and the types of deficiencies identified. The quality evaluation may also indicate whether corrective action should be taken for the entity being evaluated based on the types and/or quantity of deficiencies identified for the entity.

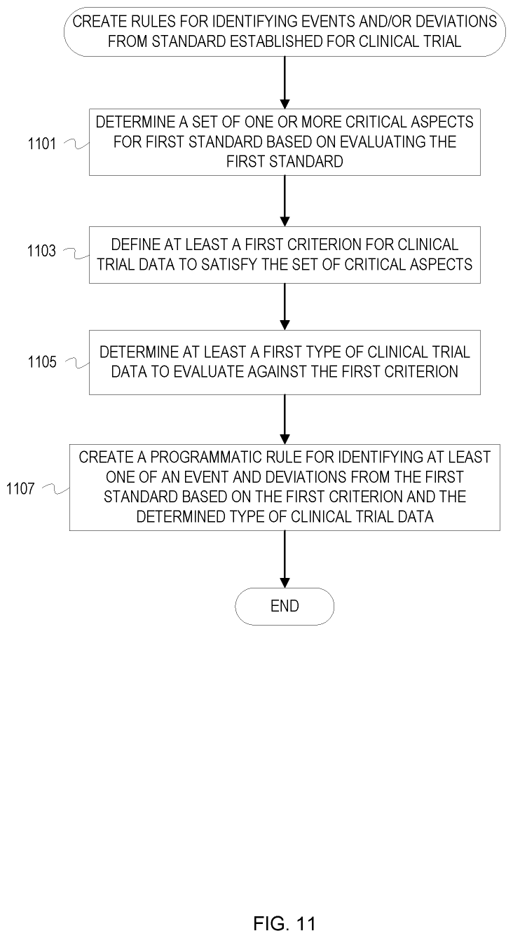

FIG. 11 is a flowchart of example operations for creating rules for identifying events and/or deviations from a standard established for a clinical trial. The example operations refer to a clinical trial reviewer as performing the depicted operations. As used herein, a clinical trial reviewer can be any entity which can generate domain knowledge input that can be leveraged for identifying protocol deviations, such as an entity which generates the domain knowledge input 405 described in reference to FIG. 4.

At block 1101, the clinical trial reviewer determines a set of one or more critical aspects for the first standard based on evaluating the first standard. The clinical trial reviewer distinguishes the critical aspects from non-critical aspects based on determining the aspects of conducting the clinical trial protocol which should be in compliance with the first standard to maintain integrity of the data collected during the clinical trial. The first standard for which the critical aspects are determined can be, for example, a clinical trial protocol, regulatory requirements, laws, other clinical trial management documents, clinical trial study plans, an expected progression of a medical condition under evaluation during the clinical trial, etc.

At block 1103, the clinical trial reviewer defines at least a first criterion for clinical trial data to satisfy the set of critical aspects. The first criterion can be a criterion for determining that a process for performing an assessment, collecting clinical trial data, or otherwise performing the clinical trial has been in compliance with the first standard. For instance, the first criterion can indicate a condition which the clinical trial data should satisfy in order to satisfy the first criterion. The clinical trial data which are evaluated against the first criterion can be data and/or metadata associated with conducting the clinical trial.

At block 1105, the clinical trial reviewer determines at least a first type of clinical trial data to evaluate against the first criterion. The type of clinical trial data can be determined based on the first criterion. For instance, the clinical trial reviewer can determine the first type of clinical trial data based on a determination of a clinical trial data source from which data can be retrieved for analysis against the first criterion (e.g., data which are stored in an EDC system, data which are stored in an eDiary system, etc.).

At block 1107, the clinical trial reviewer creates a programmatic rule for identifying at least one of an event and deviations from the first standard based on the first criterion and the determined type of clinical trial data. The programmatic rule indicates at least one criterion for satisfying the set of critical aspects to determine that the clinical trial data satisfy the first criterion and thus indicate that the clinical trial has been conducted in accordance with the first standard. The programmatic rule can also indicate the first type of clinical trial data to evaluate for satisfaction of the first criterion (e.g., based on the corresponding source of the first type of clinical trial data). Clinical trial data can subsequently be evaluated against the programmatic rule to identify if an event which triggers discontinuation of the clinical trial has occurred and/or if a deviation from the first standard has occurred.

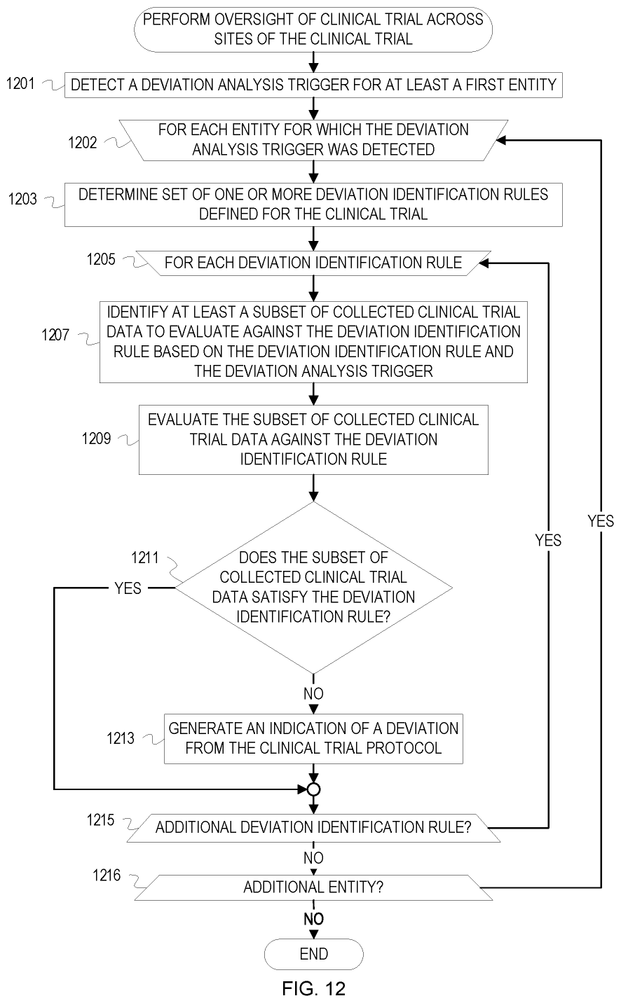

FIG. 12 is a flowchart of example operations for performing oversight of a clinical trial across sites of the clinical trial. The example operations refer to a deviation identification system as performing the example operations for consistency with FIG. 4, although naming of software and program code can vary among implementations.

At block 1201, the deviation identification system detects a deviation analysis trigger for at least a first entity. The first entity for which deviation analysis is triggered may be a first of the clinical trial sites, a study participant, a study team member, or a clinical trial management team. The trigger for deviation analysis can be based on, for example, an expiration of a time interval, creation and/or updates of records which are stored in a clinical trial data source, a schedule defined for performing deviation analysis, etc.

At block 1202, the deviation identification system begins deviation identification for each entity for which the deviation analysis trigger was detected. At block 1203, the deviation identification system determines a set of one or more deviation identification rules defined for the clinical trial. One or more rules for deviation identification can be attached to the deviation identification system. The rules can indicate at least a first critical aspect for compliance with a protocol associated with the clinical trial and conditions for the clinical trial data to satisfy the first critical aspect.

At block 1205, the deviation identification system begins evaluating clinical trial data against each deviation identification rule. The deviation identification system evaluates clinical trial data against the deviation identification rules in the set of one or more deviation identification rules defined for the clinical trial.

At block 1207, the deviation identification system identifies at least a subset of collected clinical trial data to evaluate against the deviation identification rule based on the deviation identification rule and the deviation analysis trigger. The deviation identification system can determine the subset of collected clinical trial data based on sources of clinical trial data which may be indicated in the deviation identification rule. The deviation identification system can also determine the subset of clinical trial data based on the entity ID(s) for which the deviation analysis trigger was detected. The subset of collected clinical trial data may have been collected from multiple data sources for clinical trial. The collected clinical trial data may be arranged by entity ID.

At block 1209, the deviation identification system evaluates the subset of collected clinical trial data against the deviation identification rule. The deviation identification system evaluates at least a first data field of the collected clinical trial data to determine if a value(s) of the data field satisfies the first critical aspect for compliance with the clinical trial protocol indicated in the deviation identification rule. For instance, the deviation identification system can compare the value(s) of the data field with a state or history maintained for the deviation identification rule which indicates one or more values which have been determined to conform to the deviation identification rule (e.g., based on past deviation analysis events).

At block 1211, the deviation identification system determines if the subset of collected clinical trial data satisfies the deviation identification rule. The deviation identification system can determine if the subset of collected clinical trial data satisfies the deviation identification rule based on whether the clinical trial data satisfy the first critical aspect established for the clinical trial protocol, such as whether the clinical trial data comprise a value(s) which matches a value(s) indicated in the state or history maintained for the deviation identification rule. If the subset of collected clinical trial data satisfies the deviation identification rule, operations continue at block 1215. If the subset of collected clinical trial data does not satisfy the deviation identification rule, operations continue at block 1213.

At block 1213, the deviation identification system generates an indication of a deviation from the clinical trial protocol. For instance, the deviation identification system can generate a notification which indicates the deviation from the clinical trial protocol, the entity ID(s) associated with the deviation, etc. The indication may be communicated to the respective study site(s) for which the deviation was identified.

At block 1215, the deviation identification system determines if an additional deviation identification rule remains for evaluation. If an additional deviation identification rule remains, operations continue at block 1205. If no additional deviation identification rules remain, operations continue at block 1216.

At block 1216, the deviation identification system determines if an additional entity remains for deviation identification. If an additional entity remains, operations continue at block 1202. If no additional entities remain, operations are complete.

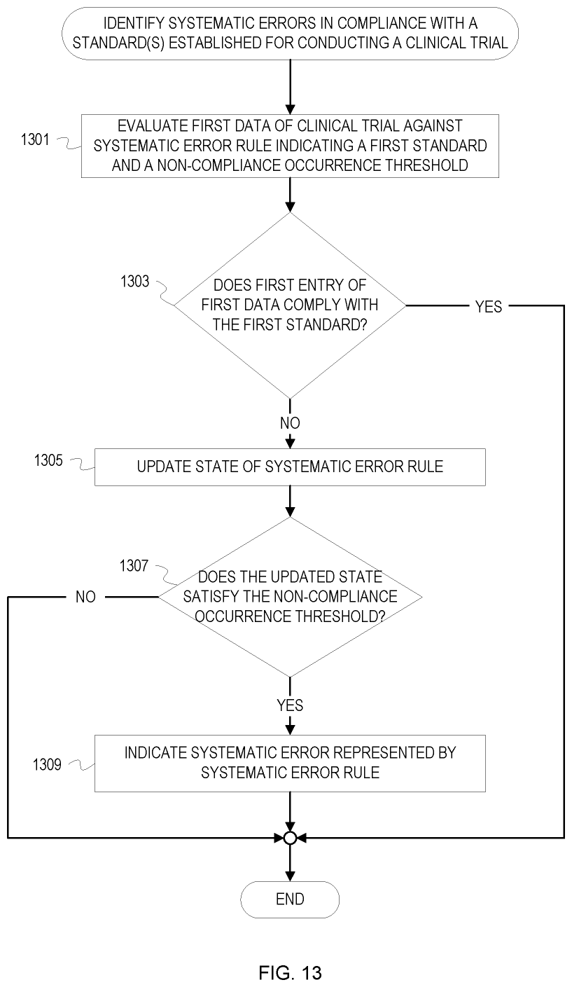

FIG. 13 is a flowchart of example operations for identifying systematic errors in compliance with one or more standards established for conducting a clinical trial. Standards established for conducting the clinical trial can include a clinical trial protocol, a study plan for the clinical trial, an expected progression of a medical condition being studied, and/or a rating of improvement of the medical condition. The example operations refer to an error identification system as performing the example operations for consistency with FIG. 5, although naming of software and program code can vary among implementations.

At block 1301, the error identification system evaluates first data of a clinical trial against a systematic error rule. The systematic error rule at least indicates a first standard for conducting the clinical trial and a non-compliance occurrence threshold. For instance, the indication of the first standard may be a critical aspect for satisfying the first standard. As an example, with reference to FIG. 5, a systematic error rule can be established for evaluating investigator consistency according to a protocol established for the clinical trial with a non-compliance occurrence threshold of 3. The first data which are evaluated against the systematic error rule may be collected from multiple sources of clinical trial data, such as a learning management system, clinical trial management system, EDC system, etc., or any combination thereof. The first data may have already been flagged as comprising a deviation from the first standard based on the first data failing to satisfy a rule for clinical trial data (e.g., as described in reference to FIG. 7).

At block 1303, the error identification system determines if a first entry of the first data complies with the first standard indicated by the systematic error rule. The error identification system can determine if the first entry complies with the first standard based on a rule for compliance with the first standard, such as based on comparing the first entry against a rule indicating a value(s) which complies with the first standard or a critical aspect associated with the first standard. As an example, with reference to FIG. 5, a rule for satisfying the standard of investigator consistency may indicate an investigator ID expected to be associated with clinical trial data collected during a particular assessment. The error identification system can determine if the first entry complies with the first standard based on determining if the first entry satisfies the rule for complying with the first standard (e.g., by determining if the first entry indicates a correct investigator ID). If the first entry of the first data does not comply with the first standard, operations continue at block 1305. If the first entry of the first data complies with the first standard, operations are complete.

At block 1305, the error identification system updates state of the systematic error rule. State can be maintained for the systematic error rule which indicates detected occurrences of non-compliance with the first standard during an error analysis. The error identification system updates the state to indicate that the first entry of the first data were indicative of a failure to comply with the first standard. The error identification system can update the state by incrementing a counter maintained for the systematic error rule which indicates the number of detected occurrences of non-compliance.

At block 1307, the error identification system determines if the updated state satisfies the non-compliance occurrence threshold. The non-compliance occurrence threshold maintained for the systematic error rule can indicate a cumulative number of detected occurrences of non-compliance with the systematic error rule which triggers detection of a systematic error. For instance, the error identification system can determine that the updated state satisfies the non-compliance occurrence threshold if the updated number of detected occurrences of non-compliance reflected in the counter maintained for the systematic error rule satisfies the threshold. If the updated state satisfies the non-compliance occurrence threshold, operations continue at block 1309. If the updated state does not satisfy the non-compliance occurrence threshold, operations are complete.

At block 1309, the error identification system indicates a systematic error represented by the systematic error rule. The systematic error rule represents a systematic error, or an error which is detected based on repeated occurrences of non-compliance with the first standard for which the rule was established. Returning to the previous example, upon determining that the updated state established for the systematic error rule for investigator consistency satisfies the non-compliance occurrence threshold of three, the error identification system can indicate that a systematic error in compliance with the rule for investigator consistency has occurred. The error identification system may also indicate the corresponding study site(s), study team member(s), participant(s), clinical trial management team(s), and/or other study site personnel for which the systematic error was detected (e.g., based on an ID(s) associated with the first data). The error identification system can guide the subsequent workflow in conducting the clinical trial based on the systematic error represented by the systematic error identification rule. For instance, the error identification system can indicate the study site(s) for which the systematic error was detected such that an onsite monitoring visit can be scheduled. As another example, the error identification system can indicate an action(s) to perform to address the systematic error (e.g., a training item to complete to remediate the cause of the systematic error). The error identification system can indicate the systematic error by generating a notification which indicates the systematic error. The error identification system may communicate the notification to the respective study site(s), study site personnel, etc. indicated in the notification of the systematic error.

Variations

While the example operations refer to detecting deviations from a clinical trial protocol based on analysis of collected clinical trial data, other standards associated with conducting a clinical trial may be established for generation of rules and analysis of the clinical trial data alternatively or in addition to the protocol. For instance, rules for deviation identification may be established based on an expected progression of a medical condition being studied during the clinical trial, patterns which are consistent with progression or treatment of the medical condition, etc. Clinical trial data can be evaluated based on the rules corresponding to the expected patterns of the medical condition to determine whether clinical trial data which have been collected for one or more participants are inconsistent with patterns of the medical condition, which may be indicative of an error in conducting the clinical trial. Clinical trial data which are inconsistent with progression or treatment of the medical condition based on the evaluation of clinical trial data against the rules can then be identified as deviations.

The flowcharts are provided to aid in understanding the illustrations and are not to be used to limit scope of the claims. The flowcharts depict example operations that can vary within the scope of the claims. Additional operations may be performed; fewer operations may be performed; the operations may be performed in parallel; and the operations may be performed in a different order. For example, with respect to FIG. 8, the operations depicted in blocks 807 and 809 can be performed in parallel or concurrently. It will be understood that each block of the flowchart illustrations and/or block diagrams, and combinations of blocks in the flowchart illustrations and/or block diagrams, can be implemented by program code. The program code may be provided to a processor of a general purpose computer, special purpose computer, or other programmable machine or apparatus.

As will be appreciated, aspects of the disclosure may be embodied as a system, method or program code/instructions stored in one or more machine-readable media. Accordingly, aspects may take the form of hardware, software (including firmware, resident software, micro-code, etc.), or a combination of software and hardware aspects that may all generally be referred to herein as a “circuit,” “module” or “system.” The functionality presented as individual modules/units in the example illustrations can be organized differently in accordance with any one of platform (operating system and/or hardware), application ecosystem, interfaces, programmer preferences, programming language, administrator preferences, etc.

Any combination of one or more machine readable medium(s) may be utilized. The machine readable medium may be a machine readable signal medium or a machine readable storage medium. A machine readable storage medium may be, for example, but not limited to, a system, apparatus, or device, that employs any one of or combination of electronic, magnetic, optical, electromagnetic, infrared, or semiconductor technology to store program code. More specific examples (a non-exhaustive list) of the machine readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a machine readable storage medium may be any tangible medium that can contain, or store a program for use by or in connection with an instruction execution system, apparatus, or device. A machine readable storage medium is not a machine readable signal medium.

A machine readable signal medium may include a propagated data signal with machine readable program code embodied therein, for example, in baseband or as part of a carrier wave. Such a propagated signal may take any of a variety of forms, including, but not limited to, electro-magnetic, optical, or any suitable combination thereof. A machine readable signal medium may be any machine readable medium that is not a machine readable storage medium and that can communicate, propagate, or transport a program for use by or in connection with an instruction execution system, apparatus, or device.

Program code embodied on a machine readable medium may be transmitted using any appropriate medium, including but not limited to wireless, wireline, optical fiber cable, RF, etc., or any suitable combination of the foregoing.

The program code/instructions may also be stored in a machine readable medium that can direct a machine to function in a particular manner, such that the instructions stored in the machine readable medium produce an article of manufacture including instructions which implement the function/act specified in the flowchart and/or block diagram block or blocks.

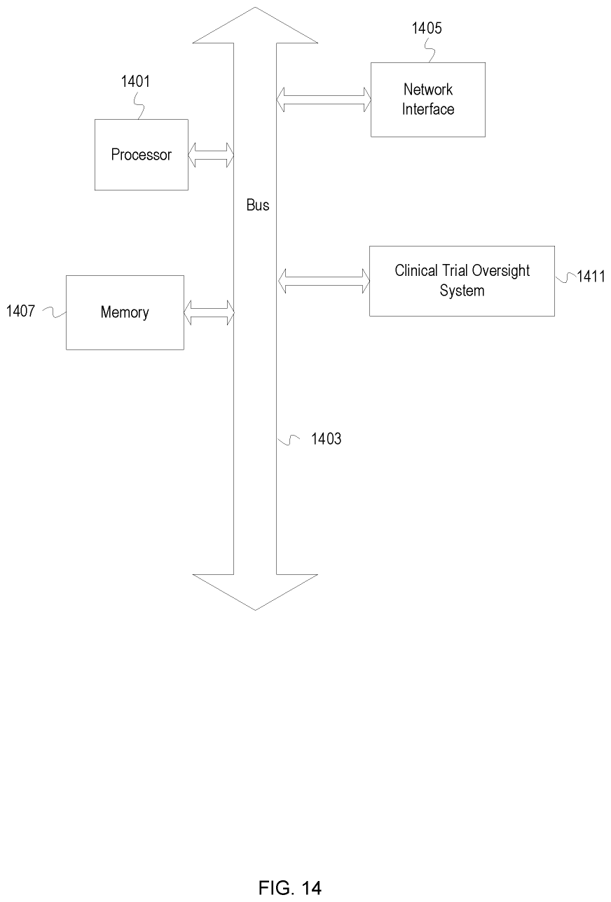

FIG. 14 depicts an example computer system with a clinical trial oversight system. The computer system includes a processor 1401 (possibly including multiple processors, multiple cores, multiple nodes, and/or implementing multi-threading, etc.). The computer system includes memory 1407. The memory 1407 may be system memory or any one or more of the above already described possible realizations of machine-readable media. The computer system also includes a bus 1403 and a network interface 1405 (e.g., wired interface, wireless interface, etc.). The system also includes clinical trial oversight system 1411. The clinical trial oversight system 1411 facilitates oversight of a clinical trial based on a protocol which has been established for the clinical trial by detecting protocol deviations, identifying systematic errors in conducting the clinical trial based on the detected protocol deviations, and performing quality evaluation of entities involved in conducting the clinical trial. Any one of the previously described functionalities may be partially (or entirely) implemented in hardware and/or on the processor 1401. For example, the functionality may be implemented with an application specific integrated circuit, in logic implemented in the processor 1401, in a co-processor on a peripheral device or card, etc. Further, realizations may include fewer or additional components not illustrated in FIG. 14 (e.g., video cards, audio cards, additional network interfaces, peripheral devices, etc.). The processor 1401 and the network interface 1405 are coupled to the bus 1403. Although illustrated as being coupled to the bus 1403, the memory 1107 may be coupled to the processor 1401.

While the aspects of the disclosure are described with reference to various implementations and exploitations, it will be understood that these aspects are illustrative and that the scope of the claims is not limited to them. In general, techniques for clinical trial monitoring and oversight as described herein may be implemented with facilities consistent with any hardware system or hardware systems. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” can be infringed with only one of the listed items, multiple of the listed items, and one or more of the items in the list and another item not listed.

1. A method comprising:
based on evaluating at least a first standard established for a clinical trial, determining a set of one or more critical aspects for the first standard;
defining at least a first criterion for clinical trial data to satisfy the set of critical aspects for the first standard, wherein the clinical trial data comprise at least one of data and metadata associated with a process for conducting the clinical trial;
determining at least a first type of clinical trial data to evaluate against the first criterion; and
creating a programmatic rule for identifying at least one of an event or deviations from the first standard based, at least in part, on the first criterion and the determined type of clinical trial data.

2. The method of claim 1, wherein defining the first criterion comprises defining a first analysis condition for satisfying the first criterion, wherein the first analysis condition is a condition for analysis of the clinical trial data of the determined type of clinical trial data.

3. The method of claim 1, wherein determining the set of one or more critical aspects comprises distinguishing the set of critical aspects from a set of one or more non-critical aspects for the first standard.

4. The method of claim 1, further comprising identifying a plurality of data sources for the type of clinical trial data, wherein creating the programmatic rule is also based on the identified plurality of data sources.

5. The method of claim 1, wherein determining the set of one or more critical aspects comprises determining a primary efficacy endpoint associated with the first standard and determining the set of critical aspects based, at least in part, on at least one of the primary efficacy endpoint and a process for measuring the primary efficacy endpoint.

6. The method of claim 1, wherein the clinical trial data further comprises at least one of operational data, medical data, and metadata associated with the at least one of the operational data and the medical data.

7. The method of claim 1, wherein the first standard comprises at least one of a clinical trial protocol, a study plan for the clinical trial, an expected progression of a medical condition being studied, and a rating of improvement of a medical condition being studied, and wherein the event comprises an event which triggers discontinuation of the clinical trial.

8. One or more non-transitory, computer-readable media having stored thereon program code for oversight of a clinical trial across sites of the clinical trial, the program code to:
detect a deviation analysis trigger for at least a first entity, wherein the first entity comprises at least one of at least a first of the clinical trial sites, a study participant, a study team member, and a clinical trial management team;
for each entity for which the deviation analysis trigger was detected,
determine a set of one or more deviation identification rules defined for the clinical trial, wherein a deviation identification rule indicates a rule for conformance by collected clinical trial data to a set of one or more critical aspects of a protocol for the clinical trial;
for each deviation identification rule,
identify at least a subset of collected clinical trial data to evaluate against the deviation identification rule based, at least in part, on the deviation identification rule and the deviation analysis trigger, wherein the subset of collected clinical trial data is from a plurality of data sources of collected clinical trial data and wherein the subset of collected clinical trial data is arranged by at least one of site identifier, study team member identifier, and participant identifier;
evaluate the subset of collected clinical trial data against the deviation identification rule; and
based, at least in part, on a determination that the subset of collected clinical trial data does not satisfy the deviation identification rule, generate an indication of a deviation from the clinical trial protocol, wherein the indication at least identifies at least one of a corresponding one of the sites, study team members, and participants.

9. The non-transitory, computer-readable media of claim 8, wherein the program code further comprises program code to maintain a state of conformance for at least a first deviation identification rule, wherein the program code to evaluate the subset of collected clinical trial data against the first deviation identification rule comprises program code to evaluate at least a first value in the subset of collected clinical trial data against the state of conformance for the first deviation identification rule.

10. The non-transitory, computer-readable media of claim 9, wherein the program code to maintain the state of conformance comprises program code to record the first value in association with the first deviation identification rule based on a determination that the first value satisfies the first deviation identification rule.

11. The non-transitory, computer-readable media of claim 8, wherein the program code to detect a deviation analysis trigger comprises at least one of program code to detect expiration of a time interval during the clinical trial, program code to detect occurrence of a scheduled event according to the clinical trial protocol, and program code to detect a set of one or more updates to at least one of the plurality of data sources.

12. The non-transitory, computer-readable media of claim 8, wherein the program code comprises program code to identify multiple of the plurality of data sources for each deviation identification rule and to obtain the subset of collected clinical trial data from the identified data sources for evaluation of the deviation identification rule.

13. The non-transitory, computer-readable media of claim 8, wherein the program code further comprises program code to:
determine whether a deviation identification rule indicates that collected clinical trial data across more than one site is to be evaluated,
wherein the program code to identify the subset of collected clinical trial data for the deviation identification rule that indicates cross-site evaluation comprises program code to identify multiple of the sites and to identify the subset of collected clinical trial data based on identifiers of the multiple of the sites.

14. The non-transitory, computer-readable media of claim 8, further comprising program code executable by a processor to:
determine a type of the deviation from the clinical trial protocol;
determine whether the deviation satisfies at least a first criterion for identification of an error in compliance with the clinical trial protocol; and
address the error.

15. The non-transitory, computer-readable media of claim 14, wherein the program code to address the error comprises program code to:
determine if the error satisfies a criterion for development of a corrective and preventative action plan (CAPA) based, at least in part, on a frequency threshold established for the type of the deviation; and
based on a determination that the error satisfies the criterion for CAPA development, indicate that a CAPA is to be developed for the error.

16. The non-transitory, computer readable media of claim 15, wherein the program code further comprises program code to log the error as an issue based on a determination that the error does not satisfy the criterion for CAPA development.

17. The non-transitory, computer-readable media of claim 14, wherein the program code to address the error comprises program code to indicate at least a first action to perform to address the error.

18. The non-transitory, computer-readable media of claim 17, wherein the first action to perform comprises an action to be performed to remediate the error, wherein the action comprises at least one of a training item, an onsite monitoring visit to at least a first of the clinical trial sites, and a meeting with at least a first of the study team members.

19. The non-transitory, computer-readable media of claim 14, wherein the first criterion for identification of the error is determined based, at least in part, on a type of the error, wherein the type of the error comprises at least one of a systematic error and a high impact error.

20. The non-transitory, computer-readable media of claim 14, wherein the program code further comprises program code to:
based on a determination that the deviation does not satisfy the first criterion for identification of the error, update state of the first criterion, wherein the state indicates a number of detected occurrences of satisfaction of the first criterion;
determine whether the updated state satisfies a compliance occurrence threshold; and
based on a determination that the updated state satisfies a compliance occurrence threshold, indicate that the error is resolved.

21. The non-transitory, computer-readable media of claim 8, wherein the program code further comprises program code to perform a quality evaluation of at least one of the corresponding one of the sites and study team members, wherein the quality evaluation is performed based, at least in part, on evaluation of the subset of collected clinical trial data.

22. A method comprising:
evaluating first data of a clinical trial against a first systematic error rule that at least indicates a first standard corresponding to the clinical trial and a non-compliance occurrence threshold;
based on a determination that a first entry of the first data does not comply with the first standard, updating state of the first systematic error rule and determining whether the updated state satisfies the non-compliance occurrence threshold; and
indicating a systematic error represented by the first systematic error rule based on a determination that the updated state satisfies the non-compliance occurrence threshold.

23. The method of claim 22, further comprising accessing multiple data sources for the clinical trial to obtain the first data.

24. The method of claim 22, wherein updating state of the first systematic error rule comprises updating a counter that indicates a number of detected occurrences of non-compliance with the first standard.

25. The method of claim 22, wherein indicating the systematic error comprises indicating at least one of an action to perform to address the systematic error and at least one of a clinical trial site, study team member, clinical trial management team, and clinical trial participant associated with the systematic error.

26. The method of claim 22, further comprising:
evaluating the first data against a second systematic error rule that at least indicates a second standard corresponding to the clinical trial and a second non-compliance occurrence threshold for the second standard; and
indicating a systematic error represented by the second systematic error rule based on a determination that a state of the second systematic error rule satisfies the second non-compliance occurrence threshold based on evaluation of the first data against the second systematic error rule.

27. The method of claim 22, further comprising determining that a corrective and preventative action plan (CAPA) is to be developed for the systematic error, wherein indicating the systematic error comprises indicating that the CAPA is to be developed for the systematic error.

28. The method of claim 22, wherein the first standard comprises at least one of a clinical trial protocol, a study plan for the clinical trial, an expected progression of a medical condition being studied, and a rating of improvement of a medical condition being studied.
